SEQUENCE LISTING (45 sequences)

SEQ ID NO: 1 - protein, 8 aa, Artificial Sequence
  8B6 VH-CDR1
EFTFTDYY

SEQ ID NO: 2 - protein, 10 aa, Artificial Sequence
  8B6 VH-CDR2
IRNRANGYTT

SEQ ID NO: 3 - protein, 10 aa, Artificial Sequence
  8B6 VH-CDR3
ARVSNWAFDY

SEQ ID NO: 4 - protein, 12 aa, Artificial Sequence
  8B6 VL-CDR1
QSLLKNNGNTFL

SEQ ID NO: 5 - protein, 3 aa, Artificial Sequence
  8B6 VL-CDR2
KVS

SEQ ID NO: 6 - protein, 9 aa, Artificial Sequence
  8B6 VL-CDR3
SQSTHIPYT

SEQ ID NO: 7 - protein, 112 aa, Artificial Sequence
  SITE 1..1: X=D or E
  Humanized anti-OAcGD2 VL consensus sequence
  SITE 2..2: X=V or I
  SITE 9..9: X=L or A
  SITE 10..10: X=S or T
  SITE 12..12: X=P or S
  SITE 13..13: X=V or L or A
  SITE 14..14: X=S or T
  SITE 15..15: X=L or P or V
  SITE 17..17: X=D or Q or E
  SITE 18..18: X=Q or P or R
  SITE 19..19: X=A or V
  SITE 20..20: X=S or T
  SITE 21..21: X=I or L
  SITE 22..22: X=S or T
  SITE 25..25: X=S or A
  SITE 29..29: X=L or V
  SITE 30..30: X=L or V
  SITE 32..32: X=N or S
  SITE 33..33: X=N or Q
  SITE 34..34: X=G or A or S
  SITE 35..35: X=N or Y or S
  SITE 36..36: X=T or N or S
  SITE 37..37: X=F or Y
  SITE 39..39: X=H or N or S or A or Y or G
  SITE 41..41: X=Y or F
  SITE 42..42: X=L or Q
  SITE 44..44: X=K or R
  SITE 47..47: X=Q or K
  SITE 48..48: X=S or A or V
  SITE 50..50: X=K or Q or R
  SITE 51..51: X=L or R or V
  SITE 55..55: X=K or G or L
  SITE 56..56: X=V or A or G
  SITE 58..58: X=N or T
  SITE 60..60: X=L or D or A
  SITE 61..61: X=S or T
  SITE 63..63: X=V or I
  SITE 65..65: X=D or A or S
  SITE 75..75: X=Y or D
  SITE 79..79: X=K or T
  SITE 81..81: X=S or N
  SITE 82..82: X=R or S
  SITE 83..83: X=V or L
  SITE 84..84: X=E or Q
  SITE 85..85: X=A or P
  SITE 88..88: X=L or V or F
  SITE 89..89: X=G or A
  SITE 90..90: X=V or T
  SITE 92..92: X=F or Y
  SITE 94..94: X=S or M or Q
  SITE 96..96: X=S or A
  SITE 97..97: X=T or Y
  SITE 98..98: X=H or Q or N
  SITE 99..99: X=I or T or Q
  SITE 100..100: X=P or S
  SITE 105..105: X=G or Q
XXVMTQSPXXLXXXXGXXXXXXCRXSQSXXKXXXXXXLXWXXQXPGXXPXXLIYXXSXRXXGXPXRFSGSGSGTXFTLXIXXXXXEDXXXYXCXQXXXXXYTFGXGTKVEIK

SEQ ID NO: 8 - protein, 119 aa, Artificial Sequence
  SITE 1..1: X=E or Q
  Humanized anti-OAcGD2 VH consensus sequence
  SITE 5..5: X=V or L
  SITE 13..13: X=Q or K
  SITE 16..16: X=G or R
  SITE 23..23: X=A or T
  SITE 24..24: X=T or A
  SITE 26..26: X=E or G
  SITE 30..30: X=T or S or G
  SITE 32..32: X=Y or H
  SITE 35..35: X=T or H or N or S
  SITE 37..37: X=V or I
  SITE 48..48: X= L or V
  SITE 49..49: X= G or S
  SITE 50..50: X= F or Y
  SITE 51..51: X= I or T
  SITE 54..54: X= R or K or S
  SITE 55..55: X= A or S
  SITE 56..56: X=N or S
  SITE 57..57: X= G or A or S
  SITE 58..58: X= Y or G
  SITE 59..59: X= T or I
  SITE 60..60: X= T or I
  SITE 61..61: X= E or Y
  SITE 63..63: X= N or A
  SITE 64..64: X= P or A or D
  SITE 76..76: X= N or G
  SITE 77..77: X= S or A
  SITE 79..79: X= S or N
  SITE 80..80: X= I or S or T
  SITE 81..81: X= L or T or A
  SITE 89..89: X= R or K or Q
  SITE 90..90: X= T or A
  SITE 95..95: X= V or I or L
  SITE 99..99: X= A or T
  SITE 105..105: X=A or Y
  SITE 114..114: X= T or L
  SITE 115..115: X= L or V
XVQLXESGGGLVXPGXSLRLSCXXSXFTFXDXYMXWXRQAPGKGLEWXXXXRNXXXXXXXXYXXSVKGRFTISRDXXKXXXYLQMNSLXXEDTAXYYCXRVSNWXFDYWGQGTXXTVSS

SEQ ID NO: 9 - protein, 119 aa, Artificial Sequence
  Anti-OAcGD2 VH49A
EVQLVESGGGLVQPGRSLRLSCTTSEFTFTDYYMTWVRQAPGKGLEWLGFIRNRANGYTTEYNPSVKGRFTISRDNSKSILYLQMNSLKTEDTAVYYCARVSNWAFDYWGQGTLVTVSS

SEQ ID NO: 10 - protein, 119 aa, Artificial Sequence
  Anti-OAcGD2 VH72A
EVQLVESGGGLVQPGGSLRLSCATSEFTFTDYYMTWVRQAPGKGLEWLGFIRNRANGYTTEYNPSVKGRFTISRDNSKNSLYLQMNSLKTEDTAVYYCARVSNWAFDYWGQGTLVTVSS

SEQ ID NO: 11 - protein, 119 aa, Artificial Sequence
  Anti-OAcGD2 VH49BHS
EVQLVESGGGLVQPGRSLRLSCTTSEFTFTDYYMTWVRQAPGKGLEWLGFIRNKANGYTTEYNPSVKGRFTISRDNSKSILYLQMNSLKTEDTAVYYCARVSNWAFDYWGQGTLVTVSS

SEQ ID NO: 12 - protein, 119 aa, Artificial Sequence
  Anti-OAcGD2 VH72BHNPS
EVQLVESGGGLVQPGGSLRLSCATSEFTFSDYYMTWVRQAPGKGLEWLGFIRNKANGYTTEYNPSVKGRFTISRDNSKNSLYLQMNSLKTEDTAVYYCARVSNWAFDYWGQGTLVTVSS

SEQ ID NO: 13 - protein, 112 aa, Artificial Sequence
  Anti-OAcGD2 VL30A
DVVMTQSPLSLPVTLGQPASISCRSSQSLLKNNGNTFLHWYQQRPGQSPRLLIYKVSNRLSGVPDRFSGSGSGTDFTLKISRVEAEDVGVYFCSQSTHIPYTFGGGTKVEIK

SEQ ID NO: 14 - protein, 112 aa, Artificial Sequence
  Anti-OAcGD2 VL28A
DVVMTQSPLSLPVTPGEPASISCRSSQSLLKNNGNTFLHWYLQKPGQSPQLLIYKVSNRLSGVPDRFSGSGSGTDFTLKISRVEAEDVGVYFCSQSTHIPYTFGQGTKVEIK

SEQ ID NO: 15 - protein, 112 aa, Artificial Sequence
  Anti-OAcGD2 VL28Bs01/A2
DVVMTQSPLSLPVTPGEPASISCRSSQSLLKSNANTFLHWYLQKPGQSPQLLIYKVSNRLSGVPDRFSGSGSGTDFTLKISRVEAEDVGVYYCMQSTHIPYTFGQGTKVEIK

SEQ ID NO: 16 - protein, 119 aa, Artificial Sequence
  Anti-OAcGD2 VH49BHSs
EVQLVESGGGLVQPGRSLRLSCTTSEFTFTDYYMTWVRQAPGKGLEWLGFIRNKASGYTTEYNPSVKGRFTISRDNSKSILYLQMNSLKTEDTAVYYCARVSNWAFDYWGQGTLVTVSS

SEQ ID NO: 17 - protein, 119 aa, Artificial Sequence
  Anti-OAcGD2 VH72BHNPSs
EVQLVESGGGLVQPGGSLRLSCATSEFTFSDYYMTWVRQAPGKGLEWLGFIRNKANSYTTEYNPSVKGRFTISRDNSKNSLYLQMNSLKTEDTAVYYCARVSNWAFDYWGQGTLVTVSS

SEQ ID NO: 18 - protein, 112 aa, Artificial Sequence
  Anti-OAcGD2 VL30As
DVVMTQSPLSLPVTLGQPASISCRSSQSLLKNQGNTFLHWYQQRPGQSPRLLIYKVSNRLSGVPDRFSGSGSGTDFTLKISRVEAEDVGVYFCSQSTHIPYTFGGGTKVEIK

SEQ ID NO: 19 - protein, 112 aa, Artificial Sequence
  Anti-OAcGD2 VL28B01/A2
DVVMTQSPLSLPVTPGEPASISCRSSQSLLKSNGNTFLHWYLQKPGQSPQLLIYKVSNRLSGVPDRFSGSGSGTDFTLKISRVEAEDVGVYYCMQSTHIPYTFGQGTKVEIK

SEQ ID NO: 20 - protein, 449 aa, Artificial Sequence
  Anti-OAcGD2 HC (anti-OAcGD2 anti-PD-L1 bispecific)
EVQLVESGGGLVQPGGSLRLSCATSEFTFSDYYMTWVRQAPGKGLEWLGFIRNKANSYTTEYNPSVKGRFTISRDNSKNSLYLQMNSLKTEDTAVYYCARVSNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 21 - protein, 219 aa, Artificial Sequence
  Anti-OAcGD2 LC (anti-OAcGD2 anti-PD-L1 bispecific)
DVVMTQSPLSLPVTLGQPASISCRSSQSLLKNNGNTFLHWYQQRPGQSPRLLIYKVSNRLSGVPDRFSGSGSGTDFTLKISRVEAEDVGVYFCSQSTHIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 452 aa, Artificial Sequence
  Anti-PD-L1 HC (anti-OAcGD2 anti-PD-L1 bispecific)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSRYTQKSLSLSPGK

SEQ ID NO: 23 - protein, 210 aa, Artificial Sequence
  Anti-PD-L1 LC (anti-OAcGD2 anti-PD-L1 bispecific)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 24 - protein, 449 aa, Artificial Sequence
  Anti-DOTA HC (anti-DOTA anti-PD-L1 bispecific)
EVMLVESGGGLVKPGGSLTLSCAASGFTFTAHAMSWVRQTPEKRLEWVATISGGGTYTYFPDSFQGRFTISSDNAKNTLYLQMSSLRSEDTSMYFCTRHGDYRYAFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 25 - protein, 216 aa, Artificial Sequence
  Anti-DOTA LC (anti-DOTA anti-PD-L1 bispecific)
QAVTQESALTTSPGETVTLTCRSSSGAVTTNNYANWVQEKPDHLFTGLIGGTNNRAPGVPARFSGSLIGDKAALTITGAQTEDEAIYFCALWFSDHWVFGGGTELTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 452 aa, Artificial Sequence; identical to SEQ ID NO: 22
  Anti-PD-L1 HC (anti-DOTAanti-PD-L1 bispecific)

SEQ ID NO: 27 - protein, 210 aa, Artificial Sequence; identical to SEQ ID NO: 23
  Anti-PD-L1 LC (anti-DOTA anti-PD-L1 bispecific)

SEQ ID NO: 28 - protein, 449 aa, Artificial Sequence; identical to SEQ ID NO: 20
  Anti-OAcGD2 HC (anti-OAcGD2 anti-DOTA bispecific)

SEQ ID NO: 29 - protein, 219 aa, Artificial Sequence; identical to SEQ ID NO: 21
  Anti-OAcGD2 LC (anti-OAcGD2 anti-DOTA bispecific)

SEQ ID NO: 30 - protein, 453 aa, Artificial Sequence
  Anti-DOTA HC (anti-OAcGD2 anti-DOTA bispecific)
EVMLVESGGGLVKPGGSLTLSCAASGFTFTAHAMSWVRQTPEKRLEWVATISGGGTYTYFPDSFQGRFTISSDNAKNTLYLQMSSLRSEDTSMYFCTRHGDYRYAFGYWGQGTLVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSRYTQKSLSLSPGK

SEQ ID NO: 31 - protein, 212 aa, Artificial Sequence
  Anti-DOTA LC (anti-OAcGD2 anti-DOTA bispecific)
QAVTQESALTTSPGETVTLTCRSSSGAVTTNNYANWVQEKPDHLFTGLIGGTNNRAPGVPARFSGSLIGDKAALTITGAQTEDEAIYFCALWFSDHWVFGGGTELTVLGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 32 - protein, 449 aa, Artificial Sequence
  Anti-OAcGD2 HC (monospecific)
EVQLVESGGGLVQPGGSLRLSCATSEFTFSDYYMTWVRQAPGKGLEWLGFIRNKANSYTTEYNPSVKGRFTISRDNSKNSLYLQMNSLKTEDTAVYYCARVSNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 33 - protein, 219 aa, Artificial Sequence; identical to SEQ ID NO: 21
  Anti-OAcGD2 LC (monospecific)

SEQ ID NO: 34 - protein, 449 aa, Artificial Sequence
  Anti-DOTA HC (monospecific)
EVMLVESGGGLVKPGGSLTLSCAASGFTFTAHAMSWVRQTPEKRLEWVATISGGGTYTYFPDSFQGRFTISSDNAKNTLYLQMSSLRSEDTSMYFCTRHGDYRYAFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 216 aa, Artificial Sequence; identical to SEQ ID NO: 25
  Anti-DOTA LC (monospecific)

SEQ ID NO: 36 - protein, 448 aa, Artificial Sequence
  Anti-PD-L1 HC (monospecific)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 214 aa, Artificial Sequence
  Anti-PD-L1 LC (monospecific)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 8 aa, Artificial Sequence
  Anti-PD-L1 H-CDR1
GFTFSDSW

SEQ ID NO: 39 - protein, 8 aa, Artificial Sequence
  Anti-PD-L1 H-CDR2
ISPYGGST

SEQ ID NO: 40 - protein, 11 aa, Artificial Sequence
  Anti-PD-L1 H-CDR3
ARRHWPGGFDY

SEQ ID NO: 41 - protein, 6 aa, Artificial Sequence
  Anti-PD-L1 L-CDR1
QDVSTA

SEQ ID NO: 42 - skipped

SEQ ID NO: 43 - protein, 9 aa, Artificial Sequence
  Anti-PD-L1 L-CDR3
QQYLYHPAT

SEQ ID NO: 44 - protein, 118 aa, Artificial Sequence [sequence omitted]

SEQ ID NO: 45 - protein, 107 aa, Artificial Sequence [sequence omitted]

(19) United States
(12) Patent Application Publication
(10) Pub. No.: US 2024/0228659 A1
(43) Pub. Date: Jul. 11, 2024

(54) NEW METHOD TO IMPROVE NK CELLS CYTOTOXICITY

(71) Applicants: INSERM (INSTITUT NATIONAL DE LA SANTÉ ET DE LA RECHERCHE MÉDICALE), Paris (FR); NANTES UNIVERSITÉ, Nantes (FR); OGD2 PHARMA, Nantes (FR)

(72) Inventors: Stéphane BIRKLE, Nantes (FR); Meriem BAHRI, Nantes (FR); Sophie FOUGERAY, Nantes (FR); Jean-Marc LE DOUSSAL, C (FR); Emilie MADURA, Nantes (FR)

(73) Assignees: INSERM (INSTITUT NATIONAL DE LA SANTÉ ET DE LA RECHERCHE MÉDICALE), Paris (FR); NANTES UNIVERSITÉ, Nantes (FR); OGD2 PHARMA, Nantes (FR)

(21) Appl. No.: 18/555,459

(22) PCT Filed: Apr. 13, 2022
(86) PCT No.: PCT/EP2022/059945

§ 371 Date: Oct. 13, 2023

(30) Foreign Application Priority Data
Apr. 14, 2021 (EP) 21305492.7

(51) Int. Cl. C07K 16/30; A61K 31/203; A61K 39/00; A61K 39/395; A61P 35/00; C07K 16/28

(52) U.S. Cl. CPC C07K 16/3084; A61K 31/203; A61K 39/39558; A61P 35/00; C07K 16/2827; A61K 2039/505; C07K 2317/24; C07K 2317/31; C07K 2317/732; C07K 2317/76

(57) ABSTRACT

A combination of retinoic acid, an anti-O-acetylated disialoganglioside (OAcGD2) compound and an anti-PD1/PD-L1 compound for treating a cancer, particularly neuroblastoma, in a subject in need thereof. The inventors focused on immunotherapeutic strategies targeting OAcGD2, believing they could address critical neuropathic pain side effects associated with anti-GD2 mAb infusions. They reported mAb 8B6 targeting OAcGD2 displays antitumor activity in NB tumor models, with induction of ADCC similarly to anti-GD2 mAbs. From this, the inventors interrogated whether 13-cis-RA and retinoic acid generally, may augment anti-NB efficiency of mAb 8B6 therapy. They found cooperative interaction of 13-cis-RA and mAb 8B6 treatment in inhibiting the NB growth in vivo. However, this combination regimen also coordinates PD-1/PD-L1 upregulation, which hinders a long-term activation of NK cells and allows tumor cell to relapse. Importantly this counter therapeutic effect can be leveraged with PD1 blockade to improve the therapeutic response of the mAb 8B6+13-cis-RA regimen.

### FIELD OF THE INVENTION

The present invention relates to a combination of retinoic acid, an anti-O-acetylated disialoganglioside (OAcGD2) compound and an anti-PD1/PD-L1 compound for use in the treatment of a cancer in a subject in need thereof.

### BACKGROUND OF THE INVENTION

Neuroblastoma (NB) is a cancer of the sympathetic nervous system derived from primordial neural crest cells that accounts for 15% of all childhood deaths (1). It is characterized by a highly heterogeneous clinical behavior, ranging from spontaneous regression to rapid progression and patient death (1). With an unfavorable prognosis, high-risk NB that occurs in half of all patients is associated with metastasis and with amplification of the MYCN oncogene, the major oncogenic driver (2, 3). Currently, the treatment for these patients consists in an aggressive multimodal therapy including surgery, radiation and high dose chemotherapy followed by stem cell rescue followed by maintenance therapy consisting in 13-cis-retinoic acid (13-cis-RA) in combination with interleukin (IL-2) and the anti-disialoganglioside GD2 monoclonal antibody (mAb) (4, 5). Although this regimen is now the standard of care, both neuropathic pain induction, associated with anti-GD2 therapeutic mAb infusion, and IL-2 related toxicities, remain a clinical challenge (4, 5). In addition, a relevant proportion of patients do not profit from this therapy (4, 5). The reasons for this treatment failure are not known. Consequently, strategies to further enhance the efficacy are important to improve the patient care and the survival rate.

The anti-tumor effects of this therapy are mainly executed by natural killer (NK) lymphocytes via antibody-dependent cell-mediated cytotoxicity (ADCC) (6, 7). These cells express the FcγRIII (CD16) receptor that binds to the Fc fragment of the antibody, and triggers the NK cell-cytotoxic effector functions. Interleukine 2 (IL-2), a stimulatory cytokine that enhances the NK cells cytotoxic activity, can also contribute to the anti-tumor effect (8). In fact, NK-cell mediated killing against NB cells is subjected to modulation through intracellular signal pathways initiated by a regulated array of activating and inhibitory receptors that engage different ligands expressed on tumor cells (9). The balance between activation and inhibition signaling decides the outcome of NK cell cytolytic response to target cells. As a result, the NB sensitivity to the NK cell-mediated killing relies on both the composition and the density of activating and inhibitory ligands expressed upon tumor cells (for review 10). As such, the programmed cell death-1 receptor (PD-1) and its ligand PD-L1 have recently been implicated in the down-regulation of NK cell mediated killing (11, 12, 13).

Retinoid therapy induces differentiation and decreases proliferation of NB (14, 15), and the rationale for combining the differentiating agent 13-cis-RA to dinutuximab relies on the observation that GD2 is a marker of mature neurons (16). Surprisingly, the contribution of 13-cis-RA in the maintenance therapy represents a poorly explored topic. Some studies have shown that 13-cis-RA treatment can induce GD2 down-regulation during NB cell differentiation in vitro (16). Moreover, retinoic acid is an important molecule in immune homeostasis (17). By and of itself, retinoic acid has been shown to regulate NK cell activity and has been shown to have a dual role: while some authors suggested that retinoic acid increases anti-GD2-mAb ADCC activity mediated by resting NK cells, others indicated that it negatively regulates NK cell cytotoxicity in preclinical models (18, 19).

### SUMMARY OF THE INVENTION

The inventors focused for many years on immunotherapeutic strategies targeting the O-acetylated form of GD2 (OAcGD2), which they believed could address the critical neuropathic pain side effects associated with anti-GD2 mAb infusions. They reported previously that mAb 8B6 targeting OAcGD2 displays antitumor activity in NB tumor models, with induction of ADCC similarly to anti-GD2 mAbs (20, 21, 22). Importantly, anti-OAcGD2 mAbs do not bind to peripheral nerves contrary to anti-GD2 therapeutic antibodies (20), and, by contrast to anti-GD2 mAbs, mAb 8B6 does not induce pain sensitization in rats (22).

Based on these findings, the inventors interrogated here whether 13-cis-RA and more generally acid retinoic, may augment the anti-NB efficiency of mAb 8B6 therapy. They found a cooperative interaction of 13-cis-RA and mAb 8B6 treatment in inhibiting the NB growth in vivo. However, this combination regimen also coordinates PD-1/PD-L1 upregulation, which hinders a long-term activation of NK cells and allows tumor cell to relapse. Importantly this counter therapeutic effect can be leveraged with PD1 blockade to improve the therapeutic response of the mAb 8B6+13-cis-RA regimen. Thus the present invention relates to a combination of retinoic acid, an anti-O-acetylated disialoganglioside (OAcGD2) compound and an anti-PD1/PD-L1 compound for use in the treatment of a cancer in a subject in need thereof. Particularly, the invention is defined by its claims.

### DETAILED DESCRIPTION OF THE INVENTION

As used herein, the expression “anti-PD1/PD-L1 compound” means “an anti-PD1 compound or an anti-PD-L1 compound”. Similarly, the expression “anti-PD1/PD-L1 antibody” means “an anti-PD1 antibody or an anti-PD-L1 antibody”.

The present invention relates to a combination of retinoic acid, an anti-O-acetylated disialoganglioside (OAcGD2) compound and an anti-PD1/PD-L1 compound for use in the treatment of a cancer in a subject in need thereof.

In another embodiment, the invention relates to i) retinoic acid, ii) an anti-OAcGD2 and iii) an anti-PD1/PD-L1 compound, as a combined preparation for simultaneous, separate or sequential use in the treatment of cancer in a subject in need thereof.

In a particular embodiment, the invention relates to a combination of retinoic acid, an anti-O-acetylated disialoganglioside (OAcGD2) compound and an anti-PD1/PD-L1 compound to improve the NK cells cytotoxicity.

In another particular embodiment, the invention relates to a combination of retinoic acid, an anti-O-acetylated disialoganglioside (OAcGD2) compound and an anti-PD1/PD-L1 compound to improve the ADCC of NK cells.

As used herein, the term “retinoic acid” denotes a metabolite of vitamin A1 (all-trans-retinol) that mediates the functions of vitamin A1 required for growth and development.

According to the invention, the retinoic acid can be the 9-cis-retinoic acid (also known as alitretinoin) or the 13-cis-retinoic acid (also known as isotretinoin). Particularly, the retinoic acid is the 13-cis-retinoic acid.

As used herein the term “O-acetylated disialoganglioside” (“OAcGD2”) refers to an O-acetylated form of the disialoganglioside GD2. Alternative names for OAcGD2 include “O-acetylated-GD2 ganglioside”, “O-acetyl GD2 ganglioside” and “O-acetyl GD2”, as non-limiting examples. As used herein, the expressions “OAcGD2”, “O-acetylated-GD2 ganglioside”, “O-acetyl GD2 ganglioside” and “O-acetyl GD2” are used indifferently. OAcGD2 is a molecule expressed in cancer tissues, including human neuroblastoma and melanoma, with highly restricted expression on normal tissues, principally to the cortex and peripheral nerves in humans.

According to the invention, the cancer may be selected in the group consisting of adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, uterine cancer, cervix cancer, colon cancer, melanoma, neuroendocrine cancer, head and neck cancer, and soft tissue tumors.

In a particular embodiment, the cancer is a neuroblastoma, a glioblastoma, a small-cell lung carcinoma or a breast cancer. More particularly, the cancer is a neuroblastoma.

In a particular embodiment, the cancer is a cancer expressing OAcGD2. As used herein, the term “cancer expressing OAcGD2” refers to cancer having cells expressing the O-acetylated form of GD2 ganglioside on their surface. Typically, said cells express more than 1,000 OAcGD2 ganglioside molecules on their cell surface, preferably more than 10,000, and more preferably more than 50,000 OAcGD2 ganglioside molecules on their cell surface. Said cancer expressing the OAcGD2 ganglioside are selected from the group comprising or consisting of neuroblastoma, glioma, retinoblastoma, Ewing's family of tumors, sarcoma (i.e. rhabdomyosarcoma, osteosarcoma, leiomyosarcoma, liposarcoma, and fibrosarcoma), small cell lung cancer, breast cancer, melanoma, metastatic renal carcinoma, head and neck cancer and hematological cancers (i.e. leukemia, Hodgkin lymphoma, non-Hodgkin lymphoma and myeloma). More generally, term “cancer expressing the OAcGD2 ganglioside” refers to cancer presenting more than 10% of cells expressing the OAcGD2 ganglioside, preferably more than 15%, and still more preferably more than 20%. Preferably, said cells are Cancer Stem Cells (CSCs).

As used herein, the term “subject” denotes a mammal, such as a rodent, a feline, a canine, and a primate. Particularly, the subject according to the invention is a human. More particularly, the human suffers of a cancer. In some embodiments, a subject may be a patient, who is awaiting the receipt of, or is receiving medical care or was/is/will be the object of a medical procedure, or is monitored for the development of a disease.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

According to the invention, the term “compounds of the invention” denotes the retinoic acid, the anti-O-acetylated disialoganglioside (OAcGD2) compound and the anti-PD1/PD-L1 compound.

In a particular embodiment, the invention also relates to allogenic NK cells (or haploidentical NK cells) modified by the compounds of the invention and administered to a subject to improve the NK cytotoxicity (ADCC) against the cancerous cells of the subject (see for example Federico Sara M. et al., 2017 (29) and Modak Shakeel et al., 2018 (30)).

As used herein the term allogenic NK cells (or haploidentical NK cells) denotes NK cells from a genetically non-identical donor of the same species, or any effector cells that have been engineered to exhibit antibody-dependent cytotoxicity.

Thus, in this case, the invention also relates to allogenic NK cells treated with the retinoic acid, an anti-O-acetylated disialoganglioside (OAcGD2) compound and an anti-PD1/PD-L1 compound for use in the treatment of a cancer in a subject in need thereof.

The terms “anti-O-acetylated disialoganglioside (OAcGD2) compound” denotes any compound (molecule) which targets the OAcGD2 (gene or protein) and inhibits its function and/or its interaction with others molecules. According to the invention, the anti-OAcGD2 compound can be an antibody which directly targets the OAcGD2 and can also be a functional fragment or a derivative thereof of a specific antibody. Alternatively, the anti-OAcGD2 compound may be a nucleic acid molecule encoding an antibody or an antigen-binding fragment that binds the OAcGD2. More particularly, the nucleic acid molecule may be a DNA or RNA, e.g. a mRNA, molecule encoding an antigen-binding fragment, such as a heavy chain variable region or a light chain variable region of an antibody, that binds the OAcGD2.

The term “anti-PD1/PD-L1 compound” denotes any compound (molecule) which will block the interaction between PD1 and PD-L1 or which will inhibit the activity or the expression of PD1 and/or PD-L1.

According to the invention, the anti-PD1/PD-L1 compound can be an antibody which directly target PD1 or PD-L1 and can also be a functional fragment or a derivative thereof of a specific antibody. Alternatively, the anti-PD1/PD-L1 compound may be a nucleic acid molecule encoding an antibody or an antigen-binding fragment that binds PD1 or PD-L1. More particularly, the nucleic acid molecule may be a DNA or RNA, e.g. a mRNA, molecule encoding an antigen-binding fragment, such as a heavy chain variable region or a light chain variable region of an antibody, that binds PD1 or PD-L1.

According to the invention, anti-O-acetylated disialoganglioside (OAcGD2) compound and anti-PD1/PD-L1 compound can be a multi-specific antibody, particularly a bi-specific antibody, according to the invention or a derivative thereof.

In order to test the functionality of a putative anti-PD1/PD-L1 compound a test is necessary. For that purpose, to identify anti-PD1/PD-L1 compound, a flow cytometry test can be provided. Cell PD1/PD-L1 expression will be monitored after incubation with various concentration of the compound for different time incubation. At the end of the incubation time, cell will be collected and stained with PD1/PD-L1-specific monoclonal antibodies. Cell-bound PD1/PD-L1-specific monoclonal antibodies will be detected by flow cytometry analysis using a fluorescent-labelled secondary antibody.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immuno-specifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

As used herein, an antibody or binding fragment thereof is said to be “immunospecific”, “specific for” or to “specifically bind” an antigen if it reacts at a detectable level with said antigen, preferably with an affinity constant (KA) of greater than or equal to about 10⁵ M⁻¹, preferably greater than or equal to about 10⁶ M⁻¹, 10⁷ M⁻¹, 10⁸ M⁻¹, 5×10⁸ M⁻¹, 10⁹ M⁻¹, 5×10⁹ M⁻¹ or more.

Affinity of an antibody or binding fragment thereof for its cognate antigen is also commonly expressed as an equilibrium dissociation constant (K_{D}). an antibody or binding fragment thereof is said to be “immunospecific”, “specific for” or to “specifically bind” an antigen if it reacts at a detectable level with said antigen, preferably with a K_{D} of less than or equal to 10⁻⁵ M, preferably less than or equal to 10⁻⁶ M, 10⁻⁷ M, 5×10⁻⁸ M, 10⁻⁸ M, 5×10⁻⁹ M, 10⁻⁹ M or less.

Affinities of antibodies or binding fragment thereof can be readily determined using conventional techniques, for example, those described by Scatchard, 1949. Ann NY Acad Sci. 51:660-672. Binding properties of an antibody or binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®) or by BioLayer Interferometry (BLI).

As used herein, the term “antibody” refers to both intact immunoglobulin molecules as well as fragments thereof that include the antigen-binding site, and includes polyclonal, monoclonal, genetically engineered and otherwise modified forms of antibodies, including but not limited to chimeric antibodies, humanized antibodies, heteroconjugate, multispecific antibodies (e.g., bispecific, trispecific or tetraspecific antibodies, diabodies, tribodies, and tetrabodies) and polypeptide-Fc fusions.

The term “antibody” as used herein also refers to antibody fragment or to an antigen-binding fragment derived directly or indirectly from immunoglobulins, such as for example a portion of an antibody, such as F(ab′)2, F(ab)2, Fab′, Fab, Fv, single-chain Fvs (scFv), single-chain antibodies, disulfide-linked Fvs (sdFv), single-domain antibodies, Fd, defucosylated antibodies, fragments comprising either a VL or VH domain, fragments produced by a Fab expression library, and anti-idiotypic (anti-id) antibodies. The term “antibody fragment” includes DARTs, and diabodies, triabodies, tetrabodies, or any other synthetic or genetically engineered proteins comprising immunoglobulin variable regions that act like an antibody by binding to a specific antigen to form a complex. The term “single-chain antibodies” also includes single heavy chain variable domains of antibodies of the type that can be found in Camelid mammals commonly known as VHH.

Thus, the term “antibody” herein also refers to single chain variants including scFv fragments, VHHs, Trans-Bodies®, Affibodies®, shark single domain antibodies, single chain or Tandem diabodies (TandAb®), Anticalins®, Nanobodies®, minibodies, BiTE®s, bicyclic peptides and other alternative immunoglobulin protein scaffolds.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding.

“Fv”, as used herein, refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Diabodies”, as used herein, refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains.

Antibody binding fragments can be obtained using standard methods. For instance, Fab or F(ab′)2 fragments may be produced by protease digestion of the isolated antibodies, according to conventional techniques.

It will also be appreciated that antibodies or binding fragments thereof according to the present invention can be modified using known methods. For example, to slow clearance in vivo and obtain a more desirable pharmacokinetic profile, the antibody or binding fragment thereof may be modified with polyethylene glycol (PEG).

“Unibodies” are well-known in the art and refer to antibody fragments lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Domain antibodies” are well-known in the art and refer to the smallest functional binding units of antibodies, corresponding to the variable regions of either the heavy or light chains of antibodies.

“Single-domain antibodies” are well-known in the art and refer to antibody-derived proteins that contain the unique structural and functional properties of naturally-occurring heavy chain antibodies. These heavy chain antibodies may contain a single variable domain (V_{H}H)—one such example is Nanobodies®—, or a single variable domain (V_{H}H) and two constant domains (C_{H}2 and C_{H}3)—such as camelid antibodies-, or a single variable domain (VHH) and five constant domains (C_{H}1, C_{H}2, C_{H}3, C_{H}4 and C_{H}5)—such as shark antibodies.

In one embodiment, the antibody or binding fragment thereof according to the present invention also encompasses multispecific antibodies or binding fragments thereof, i.e., being immunospecific for more than one, such as at least two, different antigens.

Antibodies anti-OAcGD2, anti-PD1 or anti-PD-L1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against OAcGD2, PD1 or PD-L1 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-OAcGD2, anti-PD1 or anti-PD-L1 single chain antibodies. Compounds useful in practicing the present invention also include anti-OAcGD2, anti-PD1 or anti-PD-L1 antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to OAcGD2, PD1 or PD-L1.

Humanized anti-OAcGD2, anti-PD1 or anti-PD-L1 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of OAcGD2, PD1 or PD-L1 are selected.

In a particular embodiment, the anti-OAcGD2 antibody according to the invention may be an antibody as explained in the patent application WO2008043777, WO2014177271, WO2015067375 or WO2018103884.

In a particular embodiment, the anti-OAcGD2 antibody is the mouse antibody 8B6 comprising:
(a) a heavy chain wherein the variable domain comprises a H-CDR1 having a sequence set forth as SEQ ID NO: 1; a H-CDR2 having a sequence set forth as SEQ ID NO: 2; a H-CDR3 having a sequence set forth as SEQ ID NO: 3; and
(b) a light chain wherein the variable domain comprises a L-CDR1 having a sequence set forth as SEQ ID NO: 4; a L-CDR2 having a sequence set forth as SEQ ID NO: 5; a L-CDR3 having a sequence set forth as SEQ ID NO: 6.

Complementarity Determining Regions sequences of 8B6 antibody are indicated in the following Table 1:

TABLE 1
(IMGT nomenclature, which is well known in
the art (The International Immunogenetics
Information System ®, LEFRANC et al, Nucleic
Acids Research, vol. 27, p: 209-212, 1999)).

| Name | SEQ ID NO: | SEQUENCE |
|---|---|---|
| 8B6 VH-CDR1 | 1 | EFTFTDYY |
| 8B6 VH-CDR2 | 2 | IRNRANGYTT |
| 8B6 VH-CDR3 | 3 | ARVSNWAFDY |
| 8B6 VL-CDR1 | 4 | QSLLKNNGNTFL |
| 8B6 VL-CDR2 | 5 | KVS |
| 8B6 VL-CDR3 | 6 | SQSTHIPYT |

Preferably, the anti-OAcGD2 antibody is a chimeric antibody, more preferably a humanized antibody or a human antibody.

In a particular embodiment, the anti-OAcGD2 antibody is a humanized antibody.

In some embodiments, the anti-OAcGD2 antibody may be a humanized antibody derived from the mouse antibody 8B6.

In a particular embodiment, the humanized antibody of the invention has the CDRs of the 8B6 antibody.

Thus, in some embodiments, the anti-OAcGD2 antibody has a sequence comprising:
heavy chain HC-CDR1 of sequence EFTFTDYY (SEQ ID NO: 1), HC-CDR2 of sequence IRNRANGYTT (SEQ ID NO: 2), HC-CDR3 of sequence ARVSNWAFDY (SEQ ID NO: 3), and
light chain LC-CDR1 of sequence QSLLKNNGNTFL (SEQ ID NO: 4), LC-CDR2 of sequence KVS (SEQ ID NO: 5), LC-CDR3 of sequence SQSTHIPYT (SEQ ID NO: 6).

In some embodiments, the anti-OAcGD2 antibody is a humanized antibody having a sequence comprising:
a light chain variable region (VL) of sequence SEQ ID NO: 7, or a sequence having a percentage of identity of at least 85% with SEQ ID NO: 7; and/or
a heavy chain variable region (VH) of sequence SEQ ID NO: 8, or a sequence having a percentage of identity of at least 85% with SEQ ID NO: 8.

Non-limiting examples of humanized anti-OAcGD2 antibody for instance include humanized antibodies having a sequence comprising:
a heavy chain variable region (VH) sequence selected from the group consisting of SEQ ID NO: 9 (“VH49A”), SEQ ID NO: 10 (“VH72A”), SEQ ID NO: 11 (“VH49BHS”), SEQ ID NO: 12 (“VH72BHNPS”), SEQ ID NO: 16 (“VH49BHSs”), SEQ ID NO: 17 (“VH72BHNPSs”), or a variant thereof; and/or
a light chain variable region (VL) sequence selected from the group consisting of SEQ ID NO: 13 (“VL30A”), SEQ ID NO: 14 (“VL28A”), SEQ ID NO: 15 (“VL28Bs01/A2”), SEQ ID NO: 18 (“VL30As”), SEQ ID NO: 19 (“VL28B01/A2”), or a variant thereof.

An illustrative and non-limiting example of a humanized anti-OAcGD2 antibody is for instance the humanized antibody having a sequence comprising:
a heavy chain (HC) sequence of SEQ ID NO: 32, or a variant thereof; and
a light chain (LC) sequence of SEQ ID NO:33, or a variant thereof.

A “variant” or “derivative” protein is defined as having a sequence identical to at least 80%, preferably at least 85%, more preferably at least 90%, even at least 95%, 96%, 97%, 98% or 99% of the reference sequence.

The amino acid residues of the antibody of the invention could be numbered according to the IMGT numbering system. The IMGT unique numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999).; Lefranc, M.-P., Pommié, C., Ruiz, M., Giudicelli, V., Foulquier, E., Truong, L., Thouvenin-Contet, V. and Lefranc, G., “IMGT unique numbering for immunoglobulin and T cell receptor variable domains and Ig superfamily V-like domains” Dev. Comp. Immunol., 27, 55-77 (2003).). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FRI-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1,111.1, 112.2, 111.2, 112.3, 111.3, etc. (http://www.imgt.org/IMGTScientificChart/Nomenclature/IMGT-FRCDRdefinition.html).

As used herein, the term “amino acid sequence” has its general meaning and is a sequence of amino acids that confers to a protein its primary structure. According to the invention, the amino acid sequence may be modified with one, two or three conservative amino acid substitutions, without appreciable loss of interactive binding capacity. By “conservative amino acid substitution”, it is meant that an amino acid can be replaced with another amino acid having a similar side chain. Families of amino acid having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In some embodiments, the anti-PD1 compound according to the invention is an anti-PD1 antibody. Said anti-PD1 antibody may be for example an antibody as explained in the patent applications WO2015035606, WO2016092419, WO2009024531 or WO2010089411.

In some embodiments, the anti-PD-1 antibody is selected from the group comprising, but not limited to, MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), CT-011 (also known as Pidilizumab, hBAT, and hBAT-1), AMP-514 (MEDI0680), spartalizumab, tislelizumab (BGB-A317), and ezabenlimab (CAS #2249882-54-8), or an antigen-binding fragment thereof.

MDX-1106, also known as MDX-1106-04, ONO-4538 or BMS-936558, is an anti-PD-1 antibody described in U.S. Pat. No. 8,008,449 and WO2006/121168.

Merck 3745, also known as MK-3475 or SCH-900475, is an anti-PD-1 antibody described in U.S. Pat. No. 8,345,509 and WO2009/114335.

CT-011 (Pidilizumab), also known as hBAT or hBAT-1, is an anti-PD-1 antibody described in WO2009/101611.

CA-170 is a PD-1 antagonist described in WO2015033301 & WO2015033299. Other anti-PD-1 antibodies are disclosed in U.S. Pat. No. 8,609,089, US 2010028330, and/or US 20120114649.

In a preferred embodiment, the anti-PD1 antibody is Nivolumab, Pembrolizumab or Pidilizumab, or an antigen-binding fragment thereof.

In some embodiments, the anti-PD-L1 compound according to the invention is an anti-PD-L1 antibody. Said anti-PD-L1 antibody according to the invention may be for example an antibody as explained in the patent application WO2010089411, WO2013079174, WO2016000619 or WO2014055897.

In some embodiments, the anti-PD-L1 antibody is selected from the group comprising, but not limited to, durvalumab (MEDI 4376), atezolizumab (MPDL3280A), avelumab (MSB0010718C), BMS-936559 (MDX-1105), MEDI0680 (AMP-514), cemiplimab (REGN2810), toripalimab (JSOOI-PD-1), camrelizumab (SHR-1210), dostarlimab (TSR-042), cetrelimab (JNJ-63723283), FAZ053, YW243.55.S70, MPDL3280A, MDX-1105, MEDI4736, CA-170, MCLA-145, SP142, STI-A1011, STI-A1012, STI-A1010, STI-A1014, A110, KY1003, or an antigen-binding fragment thereof.

Antibody YW243.55.S70 is an anti-PD-L1 described in WO 2010/077634 A1. MEDI4736 is an anti-PD-L1 antibody described in WO2011/066389 and US2013/034559.

MDX-1105, also known as BMS-936559, is an anti-PD-L1 antibody described in WO2007/005874.

Atezolizumab is an anti-PD-L1 antibody described in U.S. Pat. No. 8,217,149.

Avelumab is an anti-PD-L1 antibody described in US 20140341917.

In a preferred embodiment, the anti-PD-L1 antibody is Avelumab, Durvalumab or Atezolizumab, or an antigen-binding fragment thereof.

In some embodiments, the anti-PD-L1 antibody for instance has a sequence comprising:
heavy chain HC-CDR1 of sequence GFTFSDSW (SEQ ID NO: 38), HC-CDR2 of sequence ISPYGGST (SEQ ID NO: 39), HC-CDR3 of sequence ARRHWPGGFDY (SEQ ID NO: 40), and
light chain LC-CDR1 of sequence QDVSTA (SEQ ID NO: 41), LC-CDR2 of sequence SAS, LC-CDR3 of sequence QQYLYHPAT (SEQ ID NO: 43).

An illustrative and non-limiting example of an anti-PD-L1 antibody is an antibody having a sequence comprising:
a light chain variable region (VL) of sequence SEQ ID NO: 44, or a variant thereof; and/or
a heavy chain variable region (VH) of sequence SEQ ID NO: 45, or a variant thereof.

In a particular embodiment, the antibodies of the invention (anti-PD1, anti-PD-L1 or anti-OAcGD2 antibodies) can also be multispecific antibodies with at least two antigen binding sites directed to OAcGD2, PD1 or PD-L1. Particularly, the multispecific antibody may be a bispecific antibody directed to OAcGD2 and PD1 or PD-L1.

As used herein, a “multispecific” binding protein or antibody is a binding protein that binds two or more antigens, and/or two or more different epitopes. A multispecific antibody that binds two antigens, and/or two different epitopes, is also referred to herein as a “bispecific” antibody. A multispecific antibody that binds three antigens, and/or three different epitopes, is also referred to herein as a “trispecific” antibody.

As used herein, the term “specificity” refers to the number of binding specificities of a binding protein, an epitope, an antigen-binding protein or an antibody. For example, the term “monospecific antibody” refers to an antibody that specifically binds to one antigen target. The term “bispecific antibody” refers to an antibody that specifically binds to two different antigen targets. The term “trispecific antibody” refers to an antibody that specifically binds to three different antigen targets. The term “tetraspecific antibody” refers to an antibody that specifically binds to four different antigen targets and so forth.

As used herein, the term “valency” refers to the number of binding sites of a binding protein, an epitope, an antigen-binding protein or an antibody. For example, the term “monovalent antibody” refers to an antibody that has one antigen-binding site. The term “bivalent antibody” or “divalent antibody” refers to an antibody that has two binding sites. The term “trivalent antibody” refers to an antibody that has three binding sites. The term “tetravalent antibody” refers to an antibody that has four binding sites. In particular embodiments, the bivalent antibody can bind to one antigen target. In other embodiments, the bivalent antibody can bind to two different antigen targets. In particular embodiments, the trivalent antibody can bind to one antigen target, i.e., is monospecific. In other embodiments, the trivalent antibody can bind to two different antigen targets, i.e., is bispecific. In other embodiments, the trivalent antibody can bind to three different antigen targets, i.e., is trispecific. In particular embodiments, the tetravalent antibody can bind to one antigen target, i.e., is monospecific. In other embodiments, the tetravalent antibody can bind to two different antigen targets, i.e., is bispecific. In other embodiments, the tetravalent antibody can bind to three different antigen targets, i.e., is trispecific. In other embodiments, the tetravalent antibody can bind to four different antigen targets, i.e., is tetraspecific.

In some embodiments, the antibody is a bispecific antibody or antibody fragment. In some embodiments, the antibody is a trispecific antibody or antibody fragment. In some embodiments, the antibody is a trispecific antibody.

In some embodiments, the multispecific antibody of the invention is a trivalent antibody comprising three antigen binding sites and collectively targeting OAcGD2 and PD1 or PD-L1.

In multispecific antibodies, multivalence targeting the same antigen, such as OAcGD2 antigen, may be necessary to conserve high binding capacity. Thus, preferably, the multispecific antibody of the invention is an at least trivalent antibody comprising at least two antigen binding sites targeting OAcGD2 and at least one antigen binding site targeting PD1 or PD-L1.

As used herein, the terms “bispecific antibody”, “bispecific Ab”, “BAb”, or the like, refer to an antibody that comprises one or two antigen(s) binding site (Fab) directed against a first antigen and one or two further binding site(s) directed against a second antigen.

The “antigen-binding sites” comprised in the multispecific antibody of the invention may any natural or engineered antigen-binding molecule such as, e.g.:
Fragment variable (Fv), including single chain fragment variable (scFv), tandem scFv, Diabodies, DART®, TandAbs, and bispecific Fv fusion antibodies with an Fc domain;
Fragment antigen binding (Fab);
   Single-domain antibodies, including Nanobodies®, VHH, V-NAR and domain antibodies (dAbs).

In some embodiments, the term “antigen-binding site” corresponds to the arms of the Y-shaped structure, which consist each of the complete light chain paired with the VH and CHI domains of the heavy chain, and are called the Fab fragments (for Fragment antigen binding).

Thus, a further aspect of the invention refers to a multispecific antibody comprising at least one first antigen-binding site that binds OAcGD2 and at least one second antigen binding site that binds PD-1 or PD-L1.

In a particular embodiment, the multispecific antibody comprises a first antigen-binding site from an anti-OAcGD2 monoclonal antibody and at least one second antigen binding site from an anti-PD-1 or anti-PD-L1 monoclonal antibody.

In some embodiments, the multispecific antibody of the invention is a bispecific antibody.

In some embodiments, the first antigen specifically bound by the multispecific antibody is OAcGD2. In some embodiments, the second antigen specifically bound by the multispecific antibody is PD-1. In some embodiments, the second antigen specifically bound by the multispecific antibody is PD-L1.

Thus, the present invention refers to a bispecific antibody comprising a first antigen-binding site that binds OAcGD2 and a second antigen-binding site that binds PD1, in particular comprising a first Fab from an anti-OAcGD2 antibody and a second Fab from an anti-PD1 antibody.

Also, the present invention refers to a bispecific antibody comprising a first antigen-binding site that binds OAcGD2 and a second antigen-binding site that binds PD-L1, in particular comprising a first Fab from an anti-OAcGD2 antibody and a second Fab from an anti-PD-L1 antibody.

In some embodiments, the antigen-binding site that binds OAcGD2 comprises the CDR or the VH/VL sequences, or fragments, variants or derivatives thereof, of the anti-OAcGD2 antibodies as described hereinabove in the present specification.

Thus, in some embodiments, the antigen-binding site that binds OAcGD2 has a sequence comprising:
heavy chain HC-CDR1 of sequence EFTFTDYY (SEQ ID NO: 1), HC-CDR2 of sequence IRNRANGYTT (SEQ ID NO: 2), HC-CDR3 of sequence ARVSNWAFDY (SEQ ID NO: 3), and
light chain LC-CDR1 of sequence QSLLKNNGNTFL (SEQ ID NO: 4), LC-CDR2 of sequence KVS (SEQ ID NO: 5), LC-CDR3 of sequence SQSTHIPYT (SEQ ID NO: 6).

In some embodiments, the antigen-binding site that binds OAcGD2 has a sequence comprising:
a light chain variable region (VL) of sequence SEQ ID NO: 7, or a sequence having a percentage of identity of at least 85% with SEQ ID NO: 7; and/or
a heavy chain variable region (VH) of sequence SEQ ID NO: 8, or a sequence having a percentage of identity of at least 85% with SEQ ID NO: 8.

Non-limiting examples of antigen-binding sites that bind OAcGD2 for instance have sequences comprising:
a heavy chain variable region (VH) sequence selected from the group consisting of SEQ ID NO: 9 (“VH49A”), SEQ ID NO: 10 (“VH72A”), SEQ ID NO: 11 (“VH49BHS”), SEQ ID NO: 12 (“VH72BHNPS”), SEQ ID NO: 16 (“VH49BHSs”), SEQ ID NO: 17 (“VH72BHNPSs”), or a variant thereof; and/or
a light chain variable region (VL) sequence selected from the group consisting of SEQ ID NO: 13 (“VL30A”), SEQ ID NO: 14 (“VL28A”), SEQ ID NO: 15 (“VL28Bs01/A2”), SEQ ID NO: 18 (“VL30As”), SEQ ID NO: 19 (“VL28B01/A2”), or a variant thereof.

In some embodiments, the antigen-binding site that binds PD-1 or PD-L1 comprises the CDR or the VH/VL sequences, or fragments, variants or derivatives thereof, of the anti-PD-1 or anti-PD-L1 antibodies as described hereinabove in the present specification.

In some embodiments, the antigen-binding site that binds PD-1 is an antigen-binding fragment of an anti-PD-1 antibody selected from the group comprising, but not limited to, MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), CT-011 (also known as Pidilizumab, hBAT, and hBAT-1), AMP-514 (MEDI0680), spartalizumab, tislelizumab (BGB-A317), and ezabenlimab (CAS #2249882-54-8), or a derivative thereof.

In some embodiments, the antigen-binding site that binds PD-L1 is an antigen-binding fragment of an anti-PD-L1 antibody selected from the group comprising, but not limited to, durvalumab (MEDI 4376), atezolizumab (MPDL3280A), avelumab (MSB0010718C), BMS-936559 (MDX-1105), MEDI0680 (AMP-514), cemiplimab (REGN2810), toripalimab (JSOOI-PD-1), camrelizumab (SHR-1210), dostarlimab (TSR-042), cetrelimab (JNJ-63723283), FAZ053, YW243.55.S70, MPDL3280A, MDX-1105, MEDI4736, CA-170, MCLA-145, SP142, STI-A1011, STI-A1012, STI-A1010, STI-A1014, A110, KY1003, or a derivative thereof.

In some embodiments, the antigen-binding site that binds PD-L1 has a sequence comprising:
heavy chain HC-CDR1 of sequence GFTFSDSW (SEQ ID NO: 38), HC-CDR2 of sequence ISPYGGST (SEQ ID NO: 39), HC-CDR3 of sequence ARRHWPGGFDY (SEQ ID NO: 40), and
light chain LC-CDR1 of sequence QDVSTA (SEQ ID NO: 41), LC-CDR2 of sequence SAS, LC-CDR3 of sequence QQYLYHPAT (SEQ ID NO: 43).

An illustrative and non-limiting example of an antigen-binding site that binds PD-L1 is an antigen-binding site having a sequence comprising:
a light chain variable region (VL) of sequence SEQ ID NO: 44, or a variant thereof; and/or
a heavy chain variable region (VH) of sequence SEQ ID NO: 45, or a variant thereof.

In a particular embodiment, a retinoic acid can be added to the multispecific antibody comprising a first antigen-binding site from an anti-OAcGD2 monoclonal antibody and at least one second antigen binding site from an anti-PD1 or anti-PD-L1 monoclonal antibody.

Exemplary formats for the multispecific antibody molecules of the invention include, but are not limited to (i) two antibodies cross-linked by chemical heteroconjugation, one with a specificity to OAcGD2 and another with a specificity to a second antigen, e.g PD1 or PD-L1; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the multispecific antibody comprises one or two Fc regions fused to one, two, three, or more antigen binding domains or other polypeptides (e.g, an Fc fusion protein).

Engineered antibodies with three or more antigen binding sites also include for example, “Octopus antibodies”, DVD-Ig, or “Dual Acting FAb”.

In some embodiments, the multispecific antibody is a dual variable domain (DVD) immunoglobulin, e.g., as described in WO2012061558. In some embodiments, the multispecific antibody comprises dual variable domains having a cross over orientation, e.g., as described in WO2012135345. In some embodiments, the multispecific antibody has a Y-shaped IgG like form, such as e.g. the “IgG configuration”, the “TBTI (tetravalent bispecific tandem immunoglobulin) configuration” or the “CODV (cross-over dual variable) configuration”).

Multispecific antibodies may also be provided in an asymmetric form with a domain crossover in one or more binding arms of the same antigen specificity, i.e. by exchanging the VH/VL domains or the complete Fab arms. In one aspect, the multispecific antibody comprises a cross-Fab fragment. The term “cross-Fab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing.

In some embodiments, the multispecific antibody comprises tandem Fabs. The term “tandem Fabs” refers to an antigen-binding protein, wherein the C terminus of one CHI region of a first Fab domain is operatively linked to the N terminus of a VH region of a second Fab domain. In certain embodiments, the tandem fab antibody may be tetravalent and monospecific (each of the four Fabs binding the same antigen). In certain embodiments, the tandem fab antibody may be tetravalent and bispecific (two of the four Fabs bind a first antigen or epitope while the other two fabs bind a second antigen or epitope).

The tandem Fabs may be operatively linked with any known peptide linker to the art used for linking two or more antigen-bind domains. In a particular embodiment, the peptide linker is a Gly-Ser linker, i.e., a linker comprising only glycine amino acid(s) and serine amino acid(s). Alternatively, or in combination with the above recited Gly-Ser linker, the peptide linker may comprise all or part of the sequence of the hinge region of one or more immunoglobulins selected from IgA, IgG, and IgD.

In some embodiments, the multispecific antibody comprises at least one pseudoFab moiety. As used herein, a “pseudoFab” moiety is analogous to a Fab moiety of a conventional antibody in that it comprises a functional antigen binding portion formed by the pairing of a variable light chain (VL) domain with a variable heavy chain (VH). However, whereas the VL and VH domains of a conventional Fab are directly fused with or linked to a constant light chain (CL) domain and a constant heavy chain 1 (CH1) domain, respectively, a pseudoFab moiety lacks CHI and CL domains. Instead, the VL and VH domains of the pseudoFab are operatively linked to a second pair of stabilized knockout VL and VH domains (denoted herein as VLX and VHX) which form an inactive or non-functional binding portion (herein, a “stabilized knockout” portion or domain) that it is incapable of specifically binding to a target antigen (e.g., any target antigen). In certain embodiments, the pseudoFab moiety is incapable of binding the target antigen of the corresponding Fab moiety from which it is derived. The pseudoFab moiety lacks CH and CL domains.

While unable to selectively bind a target antigen, the VLX and VHX domains of a pseudoFab nevertheless preferentially associate which each other to form a stable chain pairing. Therefore, by appending a pseudoFab to one or more additional binding domains of differing specificities, the inherent stability of the VLX/VHX chain pairing of a pseudoFab can drive heterodimerization of the chains of a desired multispecific binding molecule.

Accordingly, a pseudoFab of the present disclosure comprises or consists of:
a first polypeptide chain having a structure represented by the formula:
   (I) VHa-L1-VHX; or
   (II) VHb-L2-VHX
and a second polypeptide chain having a structure represented by the formula:
   (III) VLa-L3-VLX; or
   (IV) VLb-L4-VLX
wherein
   VHX associates with VLX to form a knockout domain,
   VH associates with VL to form a first functional antigen binding domain, and
   L1, L2, L3 and L4 are linkers, which may present or absent, identical or different.

Other examples of bispecific antibody formats include, but are not limited to, the so-called “BiTE” (bispecific T cell engager) molecules wherein two scFv molecules are fused by a flexible linker; diabodies and derivatives thereof, such as tandem diabodies (“TandAb”); “DART” (dual affinity retargeting) molecules which are based on the diabody format but feature a C-terminal disulfide bridge for additional stabilization, and so-called triomabs, which are whole hybrid mouse/rat IgG molecules.

Multispecific antibodies can be generated by combination of polypeptides chains targeting different antigens. The 2+1 or 2+2 antigen-binding valencies leading to tri or tetravalent molecules can be obtained from a whole IgG combined to antigen-binding building blocks derived from immunoglobulin domain of native antibodies such as single-domain antibody (SDA or VHH), variable fragment (Fv), single-chain variable fragment (scFv), Fab fragment, and single chain antigen-binding fragment, or bispecific antibody conjugates combining two whole IgG molecules linked together via a linker on Fc domain. Multispecific antibodies can also be designed with antigen-binding building blocks without the Fc domains.

Illustrative and non-limiting examples of bispecific multivalent antibody formats comprising an Fc domain include:
DVD-IgG constructs where monospecific IgG are designed for bispecificity via appending to the VL and VH domains of an IgG with one specificity, at the amino or carboxy terminus of either the light or heavy chain, with additional VL and VH of an IgG of different specificity;
scFv fusion proteins where addition of a new antigen-binding moiety (scFv) to full length IgG results in fusion proteins with tetravalency (available as C-terminal scFv fusion or N-terminal scFv fusion) leading to scFv(H)-IgG, scFv(L)-IgG, IgG(H)-scFv or IgG(L)scFv constructs;
Fab(H)-IgG where Fab is fused to the heavy chain of an IgG;
Diabody-Fc fusions where the Fab fragment of an IgG is replaced by a bispecific diabody;
scFv₄-IgG constructs containing two polypeptides, (scFv)₁-CL and (scFv)₂-CH1-CH2-CH3 leading to BsAb via the natural dimerization mechanism between IgG heavy and light chains;
IgG-scFab constructs which are engineered by fusing a scFab domain to the C-termini of IgG heavy chains modified by knobs-into-holes (KIH) technique.
DNL-Fab3 constructs which are trivalent antibodies composed of three Fab fragments joined by the use of the specific interaction between the regulatory subunits of cyclic adenosine monophosphate (cAMP)-dependent protein kinase A (PKA) and the anchoring domains of A kinase anchoring proteins (AKAP);
Fab-scFv-Fc where a Fab and a scFv are fused, separately, to the N-terminus of the heterodimerizing Fc chains.

Multispecific antibodies may be prepared as full-length antibodies or antibody fragments. Techniques for making multispecific antibodies include, but are not limited to, recombinant co-expression of two immunoglobulin heavy chain-light chain pairs having different specificities and “knob-in hole” engineering (see, e.g., U.S. Pat. No. 5,731,168). Nonlimiting exemplary knob-in-hole substitutions include T366W (knob) and T366S/L368A/Y407V (hole). In some embodiments, the knob-in-hole substitutions are in IgG1 constant domains.

Multispecific antibodies may also be made by engineering electrostatic steering effects for making antibody Fc-heterodimeric molecules. As a nonlimiting example, in a bispecific antibody comprising two heavy chain variable regions and two light chain variable regions, a first heavy chain variable region may comprise a Q39E substitution (Kabat numbering) and a first light chain variable region may comprise a Q38K substitution (Kabat numbering); and a second heavy chain variable region may comprise a Q39K substitution (Kabat numbering) and a second light chain variable region may comprise a Q38E substitution (Kabat numbering). In some embodiments, the Q39E/Q38K and Q39K/Q38E substitutions reduce mispairing of the heavy and light chains of the bispecific antibody. Similarly, a first heavy chain constant region may comprise a S183K substitution (EU numbering) and a first light chain constant region may comprise a V133E substitution (EU numbering), and a second heavy chain constant region may comprise a S183E substitution (EU numbering) and a second light chain constant region may comprise a V133K substitution (EU numbering). In some embodiments, the S183K/V133E and S183E/V133K substitutions reduce mispairing of the heavy and light chains of the bispecific antibody.

In some embodiments, a bispecific antibody comprises Q39E/Q38K and Q39K/Q38E substitutions in the binding domains and S183K/V133E and S183E/V133K substitutions in the constant regions. In some embodiments, a bispecific antibody comprises both knob-in-hole substitutions and electrostatic substitutions.

Multi-specific antibodies may also be made by cross-linking two or more antibodies or fragments; using leucine zippers to produce bi-specific antibodies (see WO 2011/034605); using the common light chain technology for circumventing the light chain mis-pairing problem (see WO 98/50431); using “diabody” technology for making bispecific antibody fragments; and using single-chain Fv (sFv) dimers.

In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is the antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is the antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl) phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such assymetrical mutations, optionally wherein one or both Fc-regions are of the IgG1 isotype.

In some embodiments, the first Fc region has an amino acid substitution at a position selected from the group consisting of: 366, 368, 370, 399, 405, 407 and 409, and the second Fc region has an amino acid substitution at a position selected from the group consisting of: 366, 368, 370, 399, 405, 407 and 409, and wherein the first and second Fc regions are not substituted in the same positions.

In some embodiments, the first Fc region has an amino acid substitution at position 405, and said second Fc region has an amino acid substitution at a position selected from the group consisting of: 366, 368, 370, 399, 407 and 409, optionally 409.

In some embodiments, the first Fc region has an amino acid substitution at position 409, and said second Fc region has an amino acid substitution at a position selected from the group consisting of: 366, 368, 370, 399, 405, and 407, optionally 405 or 368.

In some embodiments, both the first and second Fc regions are of the IgG1 isotype, with the first Fc region having a Leu at position 405, and the second Fc region having an Arg at position 409.

In some embodiments, the bispecific antibody is obtained or obtainable via a method that maintains natural Fab structures of both original mAbs as well as full human Fc, described in Golay et al, 2016 (Golay et al, Design and Validation of a Novel Generic Platform for the Production of Tetravalent IgGI-like Bispecific Antibodies. J Immunol. 2016) and WO2013005194.

In some embodiment, the bispecific antibodies of the present invention comprise Fab fragments having mutations at the interface of the CHI and CL domains, said mutations preventing heavy chain/light chain mispairing.

In some embodiment, the CHI domain of the Fab fragments has mutations selected from the group consisting in: substitution of the threonine residue at position 192 with a glutamic acid residue; substitution of the leucine residue at position 143 with a glutamine residue and substitution of the serine residue at position 188; substitution of the leucine residue at position 124 with an alanine residue and substitution of the leucine residue at position 143 with a glutamic acid residue; and substitution of the valine residue at position 190 with an alanine residue.

In some embodiment, the CL domain of the Fab fragments has mutations selected from the group consisting in: substitution asparagine residue at position 137 with a lysine residue and substitution of the serine residue at position 114 with an alanine residue; substitution of the valine residue at position 133 with a threonine residue and substitution of the serine residue at position 176 with an valine residue; substitution of the valine residue at position 133 with a tryptophane residue; and substitution of the leucine residue at position 135 with a tryptophane residue and substitution of the asparagine residue at position 137 with an alanine residue.

In some embodiment, the bispecific antibodies of the present invention comprises Fab fragments having mutations at the interface of the CHI and CL domains, said mutations preventing heavy chain/light chain mispairing and said Fab fragments being tandemly arranged in any order, the C-terminal end of the CHI domain of the first Fab fragment being linked to the N-terminal end of the VH domain of the following Fab fragment through a polypeptide linker. Generally, said polypeptide linker should have a length of at least 20, preferably at least 25, and still more preferably at least 30, and up to 80, preferably up to 60, and still more preferably up to 40 amino-acids.

Advantageously, said polypeptide linker comprises all or part of the sequence of the hinge region of one or more immunoglobulin(s) selected among IgA, IgG, and IgD.

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently.

In some embodiment, the polypeptide linker has a length of at least 20 amino-acids.

In some embodiment, the bispecific antibody of the invention has an immunoglobulin-like structure.

Furthermore, a multispecific antibody according to the invention may also be a fusion protein comprising at least two different antibody fragments or antigen-binding fragment with a specificity to OAcGD2 and another with a specificity to a second antigen, e.g PD1 or PD-L1; fused to another polypeptide, for example an Fc domain.

Furthermore, a multispecific antibody according to the invention may consist of a fusion protein comprising at least one VHH molecule with specificity to OAcGD2 and at least another with a specificity to a second antigen, e.g PD1 or PD-L1; fused to each other, with or without a linker, and/or fused to a further polypeptide, for example an Fc domain.

The anti-OAcGD2 compound may also be one or more nucleic acid molecule(s) encoding a multispecific antibody that binds OAcGD2. More particularly, said nucleic acid molecules may be DNA or RNA, e.g. mRNA, molecule(s) encoding a multispecific antibody that binds OAcGD2.

Similarly, the anti-PD1/PD-L1 compound may also be one or more nucleic acid molecule(s) encoding a multispecific antibody that binds PD1 or PD-L1. More particularly, said nucleic acid molecules may be DNA or RNA, e.g. mRNA, molecules encoding a multispecific antibody that binds PD1 or PD-L1.

The present invention also provides antibodies comprising functional variants of the VL region, VH region, or one or more CDRs of the antibodies or multi-specific antibody of the invention. A functional variant of a VL, VH, or CDR used in the context of a monoclonal antibody of the present invention still allows the antibody to retain at least a substantial proportion (at least about 50%, 60%, 70%, 80%, 85%, 90%, 95% or more) of the affinity/avidity and/or the specificity/selectivity of the parent antibody and in some cases such a monoclonal antibody of the present invention may be associated with greater affinity, selectivity and/or specificity than the parent Ab. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation. Such functional variants typically retain significant sequence identity to the parent Ab.

“Identity” or “identical”, when used herein in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

The sequence of CDR variants may differ from the sequence of the CDR of the parent antibody sequences through mostly conservative substitutions; for instance, at least about 35%, about 50% or more, about 60% or more, about 70% or more, about 75% or more, about 80% or more, about 85% or more, about 90% or more, (e.g., about 65-95%, such as about 92%, 93% or 94%) of the substitutions in the variant are conservative amino acid residue replacements. The sequences of CDR variants may differ from the sequence of the CDRs of the parent antibody sequences through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the antibodies of the invention. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 70% of identity to the parent peptide. According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the present invention a first amino acid sequence having at least 90% of identity with a second amino acid sequence means that the first sequence has 90; 91; 92; 93; 94; 95; 96; 97; 98; 99; or 100% of identity with the second amino acid sequence.

In another embodiment, the antibody according to the invention is a single domain antibody against OAcGD2, PD1 or PD-L1. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the anti-PD1/PD-L1 compound according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In one embodiment, the anti-PD1/PD-L1 compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of PD1/PD-L1 are selected.

In another embodiment, the anti-PD1/PD-L1 compound according to the invention is an inhibitor of PD1 or PD-L1 gene expression.

Small inhibitory RNAs (siRNAs) can also function as inhibitors of PD1 or PD-L1 expression for use in the present invention. PD1 or PD-L1 gene expression can be reduced by contacting a subject or cell with a small double stranded RNA (dsRNA), or a vector or construct causing the production of a small double stranded RNA, such that PD1 or PD-L1 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of PD1 or PD-L1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of PD1 or PD-L1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of PD1 or PD-L1 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing PD1 or PD-L1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In another embodiment, the invention relates to a method for treating a cancer comprising administering to a subject in need thereof a therapeutically effective amount of retinoic acid, an anti-O-acetylated disialoganglioside (OAcGD2) compound and an anti-PD1/PD-L1 compound.

In another embodiment, the invention relates to the use of combination of retinoic acid, an anti-O-acetylated disialoganglioside (OAcGD2) compound and an anti-PD1/PD-L1 compound for the manufacturing of a medicament for the treatment of a cancer in a subject in need thereof.

In another embodiment, the invention relates to a pharmaceutical composition for the treatment of a cancer in a subject in need thereof, said pharmaceutical composition comprising retinoic acid, an anti-O-acetylated disialoganglioside (OAcGD2) compound and an anti-PD1/PD-L1 compound.

According to the invention, the compounds of the invention can also be used in combination with at least one other therapeutic active agent as described below.

### Nucleic Acids, Vectors, Recombinant Host Cells and Uses Thereof

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the antibodies and polypeptides of the invention, it would be necessary to employ vectors comprising polynucleotide molecules for encoding the antibodies and polypeptides of the invention. Methods of preparing such vectors as well as producing host cells transformed with such vectors are well known to those skilled in the art.

Accordingly, a further object of the invention relates to a nucleic acid molecule encoding an antibody according to the invention. More particularly the nucleic acid molecule encodes a heavy chain or a light chain of an antibody of the present invention.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Any expression vector for animal cell can be used, so long as a gene encoding the human antibody C region can be inserted and expressed. Examples of suitable vectors include PAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The choice of a suitable expression vector for expression of the antibodies of the invention will of course depend upon the specific host cell to be used, and is within the skill of the ordinary artisan. Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., a monoclonal antibody). Thus, a promoter nucleotide sequence is operably linked to a given DNA sequence if the promoter nucleotide sequence directs the transcription of the sequence.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

### Therapeutic or Pharmaceutical Composition

Another object of the invention relates to a therapeutic or pharmaceutical composition comprising retinoic acid, an anti-O-acetylated disialoganglioside (OAcGD2) compound and an anti-PD1/PD-L1 compound according to the invention for use in the treatment of cancer in a subject in need thereof.

In some embodiment, the invention relates to a therapeutic or pharmaceutical composition comprising retinoic acid and a multi-specific antibody according to the invention for use in the treatment of cancer in a subject in need thereof.

In a particular embodiment, the invention relates to a therapeutic or pharmaceutical composition comprising retinoic acid and a bi-specific antibody comprising a first Fab from an anti-OAcGD2 antibody and a second Fab from an anti-PD1 antibody or comprising a first Fab from an anti-OAcGD2 antibody and a second Fab from an anti-PD-L antibody according to the invention for use in the treatment of cancer in a subject in need thereof.

In a particular embodiment, the invention relates to a therapeutic or pharmaceutical composition comprising retinoic acid and a fusion protein comprising at least two different antibody fragments or antigen-binding fragment with a specificity to OAcGD2 and another with a specificity to a second antigen, e.g PD1 or PD-L1; fused to another polypeptide, for example an Fc domain.

In a particular embodiment, the invention relates to a therapeutic or pharmaceutical composition comprising retinoic acid and a fusion protein comprising at least one VHH molecules with specificity to OAcGD2 and at least another with a specificity to a second antigen, e.g PD1 or PD-L1; fused to each other, with or without a linker, and/or fused to a further polypeptide, for example an Fc domain.

Any therapeutic agent of the invention may be combined with pharmaceutically acceptable excipients, and optionally sustained-release matrices, such as biodegradable polymers, to form therapeutic or pharmaceutical compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Particularly, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise at least one further therapeutic active agent. The present invention also relates to a kit comprising the compounds of the invention and a further therapeutic active agent.

For example, anti-cancer agents may be added to the pharmaceutical composition as described below.

Anti-cancer agents may be agents already used for example in neuroblastoma like interline 2 (IL-2) and antibody anti-disialoganglioside (anti-GD2). This agent can also be used in combination with the compounds of the invention.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family.

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1. 13-cis-Retinoic acid enhances anti-neuroblastoma activity of anti-O-acetylated GD2 ganglioside in vivo. (A) Mice (8 animal/group) were inoculated with 2.5×10⁵ NXS2 cells by i.v. injections on day 0. Antibody 8B6 treatment was performed on days 3, 7, 10, 14, 17, and 21 (cumulative dose=150 μg). 13-cis-retinoic acid treatment was given at 10 mg/kg, per os, five times weekly, day 17 to 21. Mice were euthanized 27 days post-tumor cell inoculation. (B) Anti-tumor efficacy was evaluated by determining the number of liver metastasis. Mean liver metastasis number±SEM of each treatment group, as depicted (** p<0.01; Mann-Whitney test). (C) The mice body weight was recorded for each treatment group, as indicated. Mean weight (mean±SEM, n=8) of mice on day 0 was defined as 100% weight. Weight in each group remained stable for the period of the treatment.

FIG. 2. Effect of retinoic acid alone or in combination with 8B6 therapy on O-acetyl-GD2 and PD-L1 expression in vivo. Tumor cells harvested from the indicated group were stained with either OAcGD2-specific mAb (A) or PD-L1-specific mAb (B). (A) Flow cytometry analysis was performed with single cell suspension obtained from the liver metastasis harvested from mice in FIG. 1 to assess the OAcGD2 expression, as indicated. The histogram chart on the left shows MFI ratio of the cells stained with mAb 8B6. The right panel shows representative flow cytometry histograms of the tumor cells stained with mAb 8B6. The level of OAcGD2 expression remains stable after treatments (ns, not significant; p>0.5). (B) Single cell suspension from (A) were also tested for PD-L1 tumor cell surface expression, as indicated. The left panel indicates the percentage of PD-L1 positive tumor cells. The right panel shows representative flow cytometry histograms of tumor cells stained with PD-L1-specific mAb. The 8B6+13-cis-RA combination therapy induces PD-L1 upregulation on the tumor cells. * p<0.05; Mann-Whitney test.

FIG. 3. 13-cis-Retinoic acid combined with mAb 8B6 increases PD-1+NK cell tumor infiltration. (A) Dot plots histograms showing the gating strategy used for flow cytometric identification of CD45+NKP46+CD3− NK cells in tumor cells. (B) Fold increase of total count of NK cells in tumor cells were achieved by flow cytometry of cells gated, as indicated. Mice treated with mAb 8B6 combined with 13-cis-RA show significant increase of tumor NK cell infiltration compared to untreated mice. (C) Percentage of PD-1+NK cells in tumor cells from the indicated group. Mice treated with mAb 8B6 combined with 13cRA show significant increase of percentage of PD-1+NK cells infiltration compared to untreated mice (*p<0.05; ** p<0.01; Mann-Whitney test).

FIG. 4. PD1 blockade increases the efficiency of the combination of 13-cis-RA with 8B6 mAb in vivo. (A) Mice (6 animal/group) were inoculated with 2.5×10⁵ NXS2 cells by IV injections on day 0. Antibody 8B6 treatment was performed on days 3, 7, 10, 14, 17, and 21 (cumulative dose=150 μg). 13-cis-retinoic acid treatment was given at 10 mg/kg, per os, five times weekly, day 17-21. Antibody RPM1-14 was administrated by i.p. twice a week at 200 μg/mice for 3 consecutive weeks. Mice were euthanized 27 days post-tumor cell inoculation. (B) Anti-tumor efficacy was evaluated by determining the liver metastasis on fresh specimen. Mean of liver metastasis number±SEM of each treatment group, as depicted (*p<0.05; ** p<0.01; *** p<0.001; Mann-Whitney test). (C) The mice body weight was recorded for each treatment group, as indicated. Mean weight (mean±SEM, n=6) of mice on day 0 was defined as 100% weight. Weight in each group remained stable for the period of the treatment.

TABLE OF
SEQUENCES:

| SEQ | | |
|---|---|---|
| ID | Sequence | |
| NO | function | Sequence |
| 1 | 8B6 mAb VH- | EFTFTDYY |
| | CDR1 | |
| 2 | 8B6 mAb VH- | IRNRANGYTT |
| | CDR2 | |
| 3 | 8B6 mAb VH- | ARVSNWAFDY |
| | CDR3 | |
| 4 | 8B6 mAb VL- | QSLLKNNGNTFL |
| | CDR1 | |
| 5 | 8B6 mAb VL- | KVS |
| | CDR2 | |
| 6 | 8B6 mAb VL- | SQSTHIPYT |
| | CDR3 | |
| 7 | Humanized | D/EV/IVMTQSPL/AS/TLP/SV/L/AS/TL/P/VGD/Q/EQ/P/RA/VS/TI/LS/TCRS/ |
| | anti-OAcGD2 | ASQSL/VL/VKN/SN/QG/A/SN/Y/ST/N/SF/YLH/N/S/A/Y/GWY/FL/QQK/RPGQ/ |
| | VL consensus | KS/A/VPK/Q/RL/R/VLIYK/G/LV/A/GSN/TRL/D/AS/TGV/IPD/A/SRFSGSGSGTY/ |
| | sequence | DFTLK/TIS/NR/SV/LE/QA/PEDL/V/FG/AV/TYF/YCS/M/QQS/AT/YH/Q/NI/T/ |
| | | QP/SYTFGG/QGTKVEIK |
| 8 | Humanized | E/QVQLV/LESGGGLVQ/KPGG/RSLRLSCA/TT/ASE/GFTFT/S/GDY/HYMT/H/ |
| | anti-OAcGD2 | N/SWV/IRQAPGKGLEWL/VG/SF/YI/TRNR/K/SA/SN/SG/A/SY/GT/IT/IE/YYN/ |
| | VH consensus | AP/A/DSVKGRFTISRDN/GS/AKS/NI/S/TL/T/AYLQMNSLR/K/QT/AEDTAV/I/ |
| | sequence | LYYCA/TRVSNWA/YFDYWGQGTT/LL/VTVSS |
| 9 | Anti-OAcGD2 | EVQLVESGGGLVQPGRSLRLSCTTSEFTFTDYYMTWVRQAPGKGLEWLGFI |
| | VH49A | RNRANGYTTEYNPSVKGRFTISRDNSKSILYLQMNSLKTEDTAVYYCARVS |
| | | NWAFDYWGQGTLVTVSS |
| 10 | Anti-OAcGD2 | EVQLVESGGGLVQPGGSLRLSCATSEFTFTDYYMTWVRQAPGKGLEWLGFI |
| | VH72A | RNRANGYTTEYNPSVKGRFTISRDNSKNSLYLQMNSLKTEDTAVYYCARVS |
| | | NWAFDYWGQGTLVTVSS |
| 11 | Anti-OAcGD2 | EVQLVESGGGLVQPGRSLRLSCTTSEFTFTDYYMTWVRQAPGKGLEWLGFI |
| | VH49BHS | RNKANGYTTEYNPSVKGRFTISRDNSKSILYLQMNSLKTEDTAVYYCARVS |
| | | NWAFDYWGQGTLVTVSS |
| 12 | Anti-OAcGD2 | EVQLVESGGGLVQPGGSLRLSCATSEFTFSDYYMTWVRQAPGKGLEWLGFI |
| | VH72BHNPS | RNKANGYTTEYNPSVKGRFTISRDNSKNSLYLQMNSLKTEDTAVYYCARVS |
| | | NWAFDYWGQGTLVTVSS |
| 13 | Anti-OAcGD2 | DVVMTQSPLSLPVTLGQPASISCRSSQSLLKNNGNTFLHWYQQRPGQSPRLL |
| | VL30A | IYKVSNRLSGVPDRFSGSGSGTDFTLKISRVEAEDVGVYFCSQSTHIPYTFGG |
| | | GTKVEIK |
| 14 | Anti-OAcGD2 | DVVMTQSPLSLPVTPGEPASISCRSSQSLLKNNGNTFLHWYLQKPGQSPQLLI |
| | VL28A | YKVSNRLSGVPDRFSGSGSGTDFTLKISRVEAEDVGVYFCSQSTHIPYTFGQ |
| | | GTKVEIK |
| 15 | Anti-OAcGD2 | DVVMTQSPLSLPVTPGEPASISCRSSQSLLKSNANTFLHWYLQKPGQSPQLLI |
| | VL28Bs01/A2 | YKVSNRLSGVPDRFSGSGSGTDFTLKISRVEAEDVGVYYCMQSTHIPYTFGQ |
| | | GTKVEIK |
| 16 | Anti-OAcGD2 | EVQLVESGGGLVQPGRSLRLSCTTSEFTFTDYYMTWVRQAPGKGLEWLGFI |
| | VH49BHSs | RNKASGYTTEYNPSVKGRFTISRDNSKSILYLQMNSLKTEDTAVYYCARVS |
| | | NWAFDYWGQGTLVTVSS |
| 17 | Anti-OAcGD2 | EVQLVESGGGLVQPGGSLRLSCATSEFTFSDYYMTWVRQAPGKGLEWLGFI |
| | VH72BHNPS | RNKANSYTTEYNPSVKGRFTISRDNSKNSLYLQMNSLKTEDTAVYYCARVS |
| | S | NWAFDYWGQGTLVTVSS |
| 18 | Anti-OAcGD2 | DVVMTQSPLSLPVTLGQPASISCRSSQSLLKNQGNTFLHWYQQRPGQSPRLL |
| | VL30As | IYKVSNRLSGVPDRFSGSGSGTDFTLKISRVEAEDVGVYFCSQSTHIPYTFGG |
| | | GTKVEIK |
| 19 | Anti-OAcGD2 | DVVMTQSPLSLPVTPGEPASISCRSSQSLLKSNGNTFLHWYLQKPGQSPQLLI |
| | VL28B01/A2 | YKVSNRLSGVPDRFSGSGSGTDFTLKISRVEAEDVGVYYCMQSTHIPYTFGQ |
| | | GTKVEIK |

### Example

### A. Efficacy of Therapeutic Combination of Retinoic Acid, Anti-OAcGD2 Antibody and Anti-PD-L1 Antibody in an Immune Competent Mouse Model of Neuroblastoma

### Material & Methods

### Therapeutic Antibodies and Other Experimental Reagents

Anti-OAcGD2 mAb 8B6 (mouse IgG2a) and an isotype control mouse IgG2a mAb generated and purified as described earlier (27). An anti-PD-1 (clone RPM1-14) was purchased from In VivoMab (BioXcell, BE0146). A BV421-conjugated mAb specific for mouse CD45 (clone 30F11), a PerCP-Cy5.5-conjugated mAb specific for mouse CD3 (clone 145-2C11), a BV421-conjugated mouse IgG control mAb (clone R35-38), and a PerCP-Cy5.5-conjugated mouse IgG control mAb (clone A19-3) were from BD Bioscience (Franklin LAkes, NJ, USA). An APC-conjugated mAb specific for mouse NKp46 (clone REA815), and an APC-conjugated mouse IgG control mAb (clone REA293) were from Miltenyi Biotec (Bergisch Gladbach, Germany). A PE-conjugated mAb specific for mouse PD-L1 (clone 10F.9G2), an APC-conjugated mAb specfic for mouse PD-1 (clone 29F.1A12), a PE-conjugated mouse IgG control antibody, and an APC-conjugated mouse IgG control antibody were from Biolegend (San Diego, CA, USA). A FITC-conjugated polyclonal antibody against mouse IgG was from Jackson Immunoresearch (Soham, UK) was used as a secondary antibody for 8B6 binding detection. A mAb specific for CD16/CD32 (clone 93) was from Invitrogen (San Diego, CA, USA). Isotretinoin—13-cis-retinoic acid—was purchased from Sigma Aldrich (Saint Louis, MO, USA).

### Cell Culture

Mouse neuroblastoma NXS2 cell line was given to us by Dr. H. N. Lode (Universitätsklinikum Greifswald, Greifswald, Germany). NXS2 cells were grown in DMEM with 10% heat-inactivated fetal calf serum, 2 mM L-Glutamin, 100 units/mL penicillin, and 100 μg/mL streptomycin, at 37° C. in 5% CO2.

### Cell Preparation and Flow Cytometry Analysis

Liver metastasis were dissociated with the mouse tumor dissociation kit and the gentle MACS dissociator (Miltenyi) according to the manufacturer's instructions. Single cell suspensions were obtained by passing through a 70 μm pore-size cell MACS SmartStrainer (Miltenyi). Red blood cells in the filtrate were lysed with RBCs lysis buffer (Stemcell, Vancouver, BC, Canada). The remaining cells were washed twice with PBS and, thereafter, stained with a Viobility 405/520 Fixable Dye stain (Miltenyi) to exclude dead cells.

For flow cytometry analysis, cells were first incubated with a mAb specific for mouse CD16/CD32 to prevent nonspecific binding of primary antibodies against FcγR, and then, were labeled with specific conjugated mAbs. Separate experiments were performed with appropriate isotype-control antibodies. Quantification of total cell numbers by flow cytometry was performed done using fluorescent beads (C36950, Thermo Fisher, Waltham, MA, USA). Flow cytometric analysis were performed using an BD FACSCanto II (BD Bioscience) and data were analyzed using FlowJo software (Flowjo LLC, Oregon, OR, USA). The fold increase of tumor NK cell infiltration was calculated by dividing the number of NK cell infiltrated of each condition by the mean of the number of NK cell.

For determination of OAcGD2 expression on tumor cell lines, cells were incubated with either mAb 8B6 or isotype control, washed with PBS, and then incubated with FITC-conjugated secondary antibody. Stained cells were subjected to flow cytometry, and data were analyzed with FlowJo software.

### Tumor Cell Engraftment and Treatments

The anti-neuroblastoma efficacy of mAb 8B6 and chemotherapeutic treatments was determined in the murine NXS2 neuroblastoma experimental liver metastasis model in A/J mice, previously described (28). NXS2 Cells (2.5×10⁵ in 100 μL of PBS) were transplanted into syngeneic 8-weeks old female A/J mice (Harlan Laboratories, Gannat, France) i.v., to induces liver metastasis 6. Mice received either OAcGD2-specific mAb 8B6 (25 μg, i.v.) or PD-1-specfic mAb RPM-14 (200 μg, i.p.) twice a week for 3 consecutive weeks beginning 3 days after tumor cell injection. Isotretinoin was given orally diluted in Ora-Plus© at 10 mg/kg daily for 5 consecutive days, starting week 3 after tumor cell challenge. Mice were euthanized on day 27 post-tumor challenge, and anti-tumor efficacy was evaluated by determining the number of liver metastasis.

### Statistical Analysis

Statistical analysis was performed using Prism software (GraphPad Prism Software). Differences between untreated and treated groups in the in vitro experiences were analyzed by Student's t test. Statistical significance of liver weights and metastasis number of experimental groups of mice was tested by two-tailed Student's t test. A p value of less than 0.05 was considered to be statistically significant. All experiment results were shown as mean±standard error of the mean (SEM).

### Study Approval

All in vivo experiments were carried out in strict accordance with the recommendations in the Guide for the Care and Use of Laboratory Animals of the French Department of Agriculture (agreement number: 00186.02). The protocol was approved by the Committee on the Ethics of Animal Experiments of the Région Pays de la Loire (permit number: 03479.01). Mice were housed at the UTE-UN animal facility (Nantes, France).

### Results

### 13-Cis-Retinoic Acid Amends mAb 8B6 Anti-Neuroblastoma In Vivo

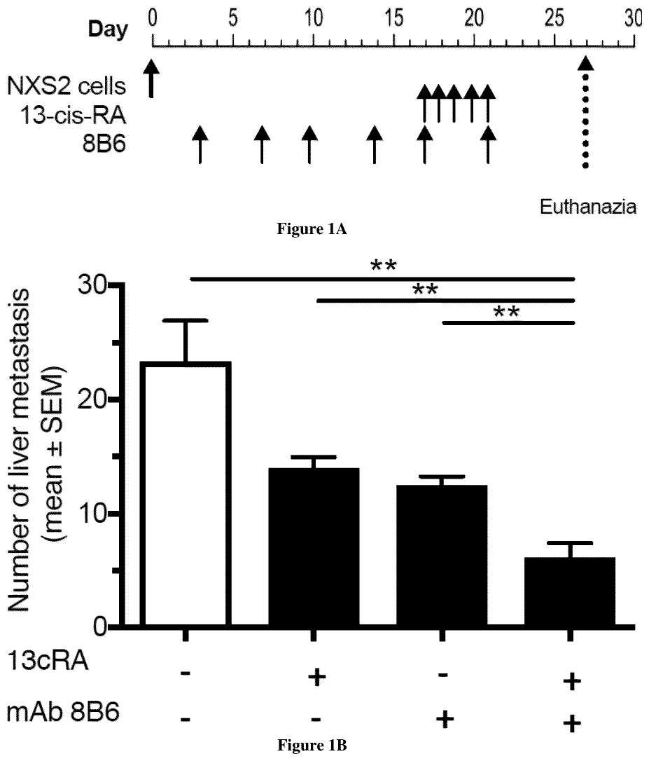

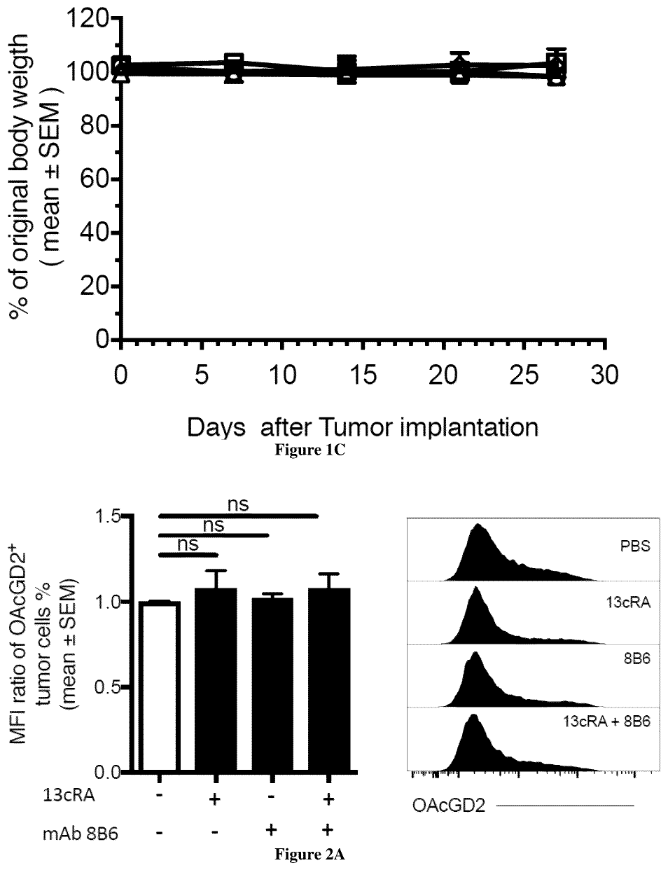

Retinoic acid administration can provide resistance to the growth of NB tumors (15). Yet, the relative contribution of 13-cis-RA to anti-NB immunotherapy remains unknown (4, 5). Therefore, we evaluated the anti-NB effect of mAb 8B6 in combination with 13-cis-RA in NXS2 NB liver metastasis bearing mice (FIG. 1). We used this model earlier to evaluate the anti-NB activity of mAb 8B6 used as a single agent (23). We calculated the 13-cis-RA dosage to reflect the human equivalent dose (23). Three days after i.v. NSX2 tumor cells inoculation, we assigned eight mice to treatment with either the single agent therapy, or the combination regimen (FIG. 1A). On day 27 after tumor cell inoculation, we determined the number of liver metastasis after mice euthanasia. The dose of 13-cis-RA (10 mg/kg, per os, Day 17 to 22) that we used here, yielded a significant reduction of NXS2 liver metastasis, as indicated by the number of liver metastasis, compared to the vehicle treated mice (13-cis-RA-treated group: 14.40±0.98, vehicle-treated group: 23.33±3.58, p<0.05; FIG. 1B). Antibody 8B6 cooperated with 13-cis-RA, resulting in a significant reduction of the mean of number of liver metastasis compared to either 13-cis-RA, or mAb 8B6 (6.83±1.16, p<0.5, FIG. 1B) used as single agent. The specificity of mAb 8B6 therapy was demonstrated, since treatment with an equivalent amount of non-specific antibody was completely ineffective (number of liver metastasis (23.33)±3.58; p>0.05 compared to vehicle-treated mice, FIG. 1B).

None of the mice did lost more than 10% of their body weight over the treatment period (FIG. 1C), demonstrating tolerability of the regimen (FIG. 1C) (26). This set of data evidences a contribution of 13-cis-RA in the in vivo anti-NB activity of the combination regimen.

O-Acetyl-GD2-Specific Antibody Plus 13-Cis-Retinoic Acid Therapy does not Impact O-Acetyl-GD2 Neuroblastoma Surface Expression

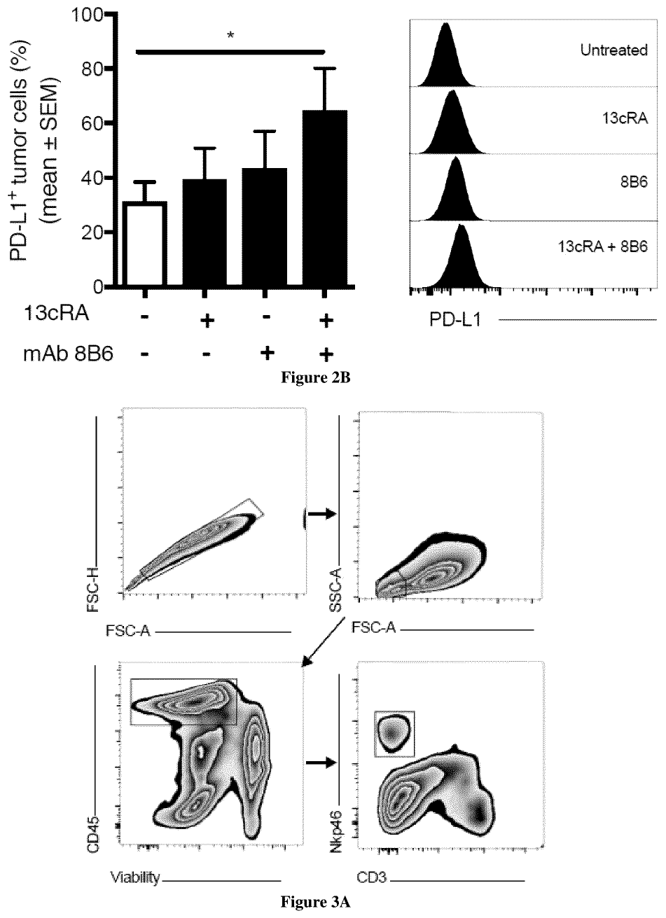

The combination showed better anti-tumor efficacy than monotherapy of either agent; it was not yet curative. Loss or down-regulation of surface antigens is a key immune mechanism of tumor relapse (25). Therefore, we collected the residual tumors of these mice to examine whether immune editing occurred after OAcGD2-specific mAb monotherapy and/or in combination with 13-cis-RA. After dissociation of the resected tumor liver metastasis, we detected OAcGD2 expression using flow cytometry analysis as described in the Material and Methods Section. The analysis of the MFI ratios indicates that the level of OAcGD2 tumor cell surface expression remains unaltered by mAb 8B6 therapy (FIG. 2A). These data suggest that OAcGD2 surface expression persists post-immunotherapy.

### 13-Cis-Retinoic Acid Impacts PD-L1 Expression in NB Cells

Another key acquired escape mechanism consists in the upregulation of the immune checkpoint PD-L1. We next examined whether PD-L1 expression was upregulated after the combination therapy. After dissociation of the resected tumor liver metastasis, we detected also PD-L1 tumor surface expression using flow cytometry analysis. We found that the percentage of PD-L1±tumor cells recovered from the untreated mice to be 31.3±7.19%. Strikingly, the combination regimen induces a significant increase in the percentage of PD-L1±tumor cells (64.62±15.49%) compared to either 13-cis-RA (39.45±9.76%) and mAb 8B6 alone (43.34±10.64%, p<0.05, FIG. 2B).

13-Cis-Retinoic Acid Combined with mAb 8B6 Induces PD-1-Expression on Intratumoral NK Cells

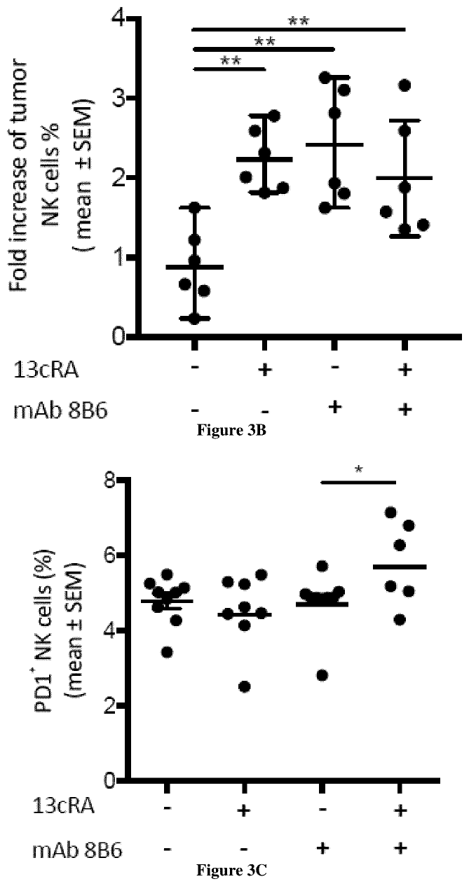

PD-L1 can inhibits NK cell activation through the engagement of PD-1 receptor (26). Consequently, we went on determining whether PD-1 receptor was expressed on NK cells infiltrating NXS2 tumors using flow cytometry analysis. The lymphocytes were identified and gated by their forward and side scatter (FIG. 3A). The NK lymphocytes were further identified and gated for the expression of CD45, NKp46, and CD3 (FIG. 3A). We found that Both 13-cis-RA and mAb 8B6 used as monotherapy induces a significant increase of intratumoral CD45+NKp46+CD3− NK cells compared to the control mice (PBS: 0.88±0.2%; 13-cis-RA: 2.49±0.29%; 8B6: 2.23±0.16%; p<0.05, FIG. 3B). Similarly, the percentage of intratumoral NK cells significantly increases in the mice that had received the combination regimen compared to the untreated mice (FIG. 3B). In this group, we further evidenced the presence of PD-1+NK cells compared to the untreated- and the monotherapy-treated mice (FIG. 3C). To sum up, these data demonstrate that an upregulation of the PD-1/PD-L1 pathway in the mice that have received the combination regimen.

PD1 Blockade Increases the Efficiency of the Combination of 13-Cis-RA with mAb 8B6 In Vivo

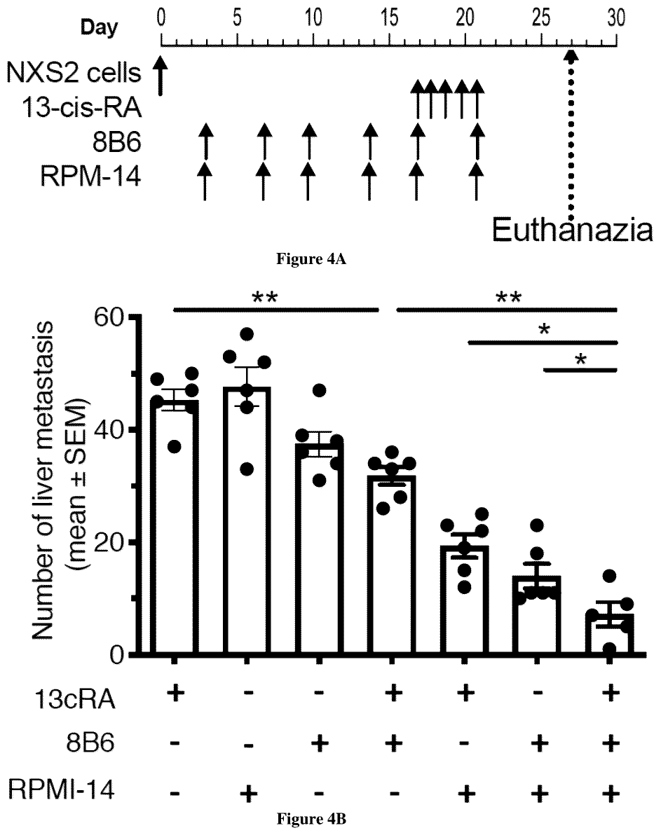

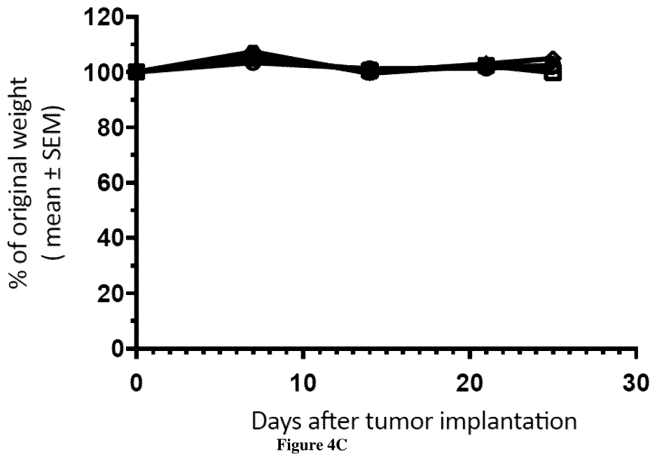

On the basis of the above results, we sought to determine whether PD-1 blockade could potentiate 13-cis-RA+8B6 combination therapy efficacy in vivo. After NXS2 challenge, we randomized mice to receive i.p. injections of saline, 13-cis-RA (10 mg/kg, per os, day 17-21), PD-1-specific mAb RPM-14 (200 μg/mice/injection, on days 3, 7, 10, 14, 17, 21, i.p.), OAcGD2-specific mAb 8B6 (25 μg/mice/injection, on days 3, 7, 10, 14, 17, 21, i.v.), mAb RPM-14+13-cis-RA, mAb 8B6+13-cis-RA, or 13-cis-RA+mAb 8B6+mAb RPM-14. Livers were harvested on day 27 and count of liver metastasis was achieved for each mouse (FIG. 4A). Mice that received PD-L1-specific mAb RPM-14 developed fewer liver metastasis compared to the control mice treated with 8B6+13-cis-RA (8B6+13-cis-RA: 31.8±3.9; 13-cis-RA+RPM-14: 19.3±5; 8B6+RPM-14: 14±5.3; 8B6+13-cis-RA+RPM-14: 7.2±4.8, FIG. 4B). Importantly, the strongest anti-NB effect is observed in the mice treated with 13-cis-RA in combination with 8B6 and RPM-14 mAbs (FIG. 4B). The statistical analysis evidences a significant tumor liver metastasis reduction compared to all other groups (FIG. 4B, p<0.05). Mice that received the combination regimen did not show any significant changes in their body weight (FIG. 4C). These data demonstrate the beneficial effect of combining PD-1 blockade to the 13-cis-RA+8B6 regimen.

### B. Efficacy of Bispecific Antibody Targeting PD-L1 and O-Acetyl GD2 Ganglioside in an Immune Competent Mouse Model of Neuroblastoma

### Material & Methods

### Design, Manufacture & Quality Control of the Bispecific Antibodies

Anti-OAcGD2/PD-L1 bispecific antibody and control antibody are synthesized as asymmetric chimeric huIgG1 grafted with the anti-muPD-L1 humanized VH from atezolizumab on one arm and the 8B6 humanized VH on the other arm. The anti-DOTA muVH (patent U.S. Pat. No. 7,230,085 B2) is used as negative control both sides.

Within the bispecific antibody, H/H heterodimerization is enriched via “knob-in-hole” mutations and the H/L pairing is directed by swapping CHI and CL domains in one arm (CrossMab technology). Control anti-OAcGD2, anti-PD-L1 and anti-DOTA huIgG1 are also synthesized. A mutation deleting protein-A binding is introduced in H chains with “holes” to eliminate mono-specific OAcGD2 antibodies.

Construct DNA is fully synthesized, cloned into pQMCF expression vector, transiently expressed in CHO cells, and purified using protein A affinity column.

### Quality Control

Purity is assessed via SDS-PAGE, analytical size-exclusion chromatography, and endotoxin tests.

Bispecific affinity is measured by BLI technology (Octet BLItZ) on immobilized purified muPDL-1 protein and OAcGD2.

### Calcein Release ADCC Assays

Human neuroblastoma LAN-1 cells are labeled with Calcein-AM for 30 min, then washed and plated onto 96-well plates at a density of 5000 cells/well in U bottom 96-well plates. Varying concentrations of the OAcGD2/PD-L1 bispecific antibody are added and peripheral blood mononuclear cells (PBMCs) are added to the LAN-1 cells wells at 1×10⁵ cells/well and incubated for 2 h (an effector:target ratio of 20:1) at 37° C. The supernatants are analyzed using fluorometry (on a Fluostar Omega Reader) to measure calcein release (cell death). For maximal release, the cells are lysed with 2% Triton X-100. The fluorescence value of the culture medium background is subtracted from that of the experimental release (A), the target cell spontaneous release (B) and the target cell maximal release (C). The cytotoxicity and ADCC percentages for each plate (in triplicate) are calculated using the following formulas:

Cytotoxicity (%)=(A−B)/(C−B)×100

ADCC (%)=Cytotoxicity (%, with antibody)−Cytotoxicity (%, without antibody)

### Mice and Tumor Models

NXS2 Cells (2.5×10⁵ in 100 μL of PBS) are transplanted into syngeneic 8-weeks old female A/J mice (Harlan Laboratories, Gannat, France) i.v., to induce liver metastasis. Mice receive 13-cis-RA, anti-OAcGD2 antibody (25 μg, i.v.), PD-L1 blocking antibody, OAcGD2/PD-L1 bispecific antibody, alone or in combination according to the invention, twice a week for 3 consecutive weeks beginning 3 days after tumor cell injection. Mice are euthanized on day 27 post-tumor challenge. Anti-tumor efficacy is evaluated by liver weight of the fresh specimen. Experiments are repeated using control bispecific antibodies.

### Results

Strong anti-NB effect is observed in the mice treated with 13-cis-RA in combination with OAcGD2/PD-L1 bispecific antibody.

### C. Anti-Tumoral Efficacy of Bispecific Antibody Targeting PD-L1 and O-Acetyl GD2 Ganglioside Assessed In Vitro in Human Cancer Models and In Vivo in an Immune-Competent Syngeneic Anti-Neuroblastoma Mouse Model

### Antibody Design and Qualification

### Design and Manufacture of the Bispecific Antibodies

Anti-OAcGD2/PD-L1 bispecific antibody was synthesized as an asymmetric huIgG1 with the humanized anti-OAcGD2 VH on one arm and the humanized anti-human PD-L1 VH from atezolizumab on the other arm. The anti-DOTA muVH (patent U.S. Pat. No. 7,230,085 B2) is used as negative control in bispecific antibody constructs (Anti-DOTA/Anti-OAcGD2 and Anti-DOTA/Anti-PD-L1). Within the bispecific antibody constructs, H/H heterodimerization is enriched via “knob-in-hole” mutations and the H/L pairing is directed by swapping CHI and CL domains in one arm (CrossMab technology, Schaefer W. et al. PNAS 2011; 108(27):11187-92). Control monospecific antibodies (anti-OAcGD2, anti-PD-L1 and anti-DOTA) are synthesized using the same “knob-in-hole” structure. A mutation deleting protein-A binding is introduced in H chains with “holes” to eliminate mono-specific OAcGD2 antibodies. Construct DNA is fully synthesized, cloned into pQMCF expression vector, transiently expressed in CHO cells, and purified using protein A affinity column.

### Antibody Sequences

The sequences of the light and heavy chains of the monospecific and bispecific antibodies are as shown in the Table below:

| SEQ | | |
|---|---|---|
| ID | Sequence | |
| NO | function | Sequence |
| 20 | Anti-OAcGD2 | EVQLVESGGGLVQPGGSLRLSCATSEFTFSDYYMTWVRQAPGKGLEWL |
| | HC | GFIRNKANSYTTEYNPSVKGRFTISRDNSKNSLYLQMNSLKTEDTAVYYC |
| | (anti-OAcGD2 | ARVSNWAFDYWGQGTLVTVSSASTKGPSVFPLAPSSKSTSGGTAALGCL |
| | anti-PD-L1 | VKDYFPEPVTVSWNSGALTSGVHTFPAVLQSSGLYSLSSVVTVPSSSLGT |
| | bispecific) | QTYICNVNHKPSNTKVDKRVEPKSCDKTHTCPPCPAPELLGGPDVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPEEKTISKAKG |
| | | QPREPQVYTLPPCREEMTKNQVSLWCLVKGFYPSDIAVEWESNGQPENN |
| | | YKTTPPVLDSDGSFFLYSKLTVDKSRWQQGNVFSCSVLHEALHSHYTQK |
| | | SLSLSPGK |
| 21 | Anti-OAcGD2 | DVVMTQSPLSLPVTLGQPASISCRSSQSLLKNNGNTFLHWYQQRPGQSPR |
| | LC | LLIYKVSNRLSGVPDRFSGSGSGTDFTLKISRVEAEDVGVYFCSQSTHIPY |
| | (anti-OAcGD2 | TFGGGTKVEIKRTVAAPSVFIFPPSDEQLKSGTASVVCLLNNFYPREAKV |
| | anti-PD-L1 | QWKVDNALQSGNSQESVTEQDSKDSTYSLSSTLTLSKADYEKHKVYACE |
| | bispecific) | VTHQGLSSPVTKSFNRGEC |
| 22 | Anti-PD-L1 HC | EVQLVESGGGLVQPGGSLRLSCAASGFTFSDSWIHWVRQAPGKGLEWV |
| | (anti-OAcGD2 | AWISPYGGSTYYADSVKGRFTISADTSKNTAYLQMNSLRAEDTAVYYCA |
| | anti-PD-L1 | RRHWPGGFDYWGQGTLVTVSSRTVAAPSVFIFPPSDEQLKSGTASVVCL |
| | bispecific) | LNNFYPREAKVQWKVDNALQSGNSQESVTEQDSKDSTYSLSSTLTLSKA |
| | | DYEKHKVYACEVTHQGLSSPVTKSFNRGECDKTHTCPPCPAPELLGGPD |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPEEKTIS |
| | | KAKGQPREPQVCTLPPSREEMTKNQVSLSCAVKGFYPSDIAVEWESNGQ |
| | | PENNYKTTPPVLDSDGSFFLVSKLTVDKSRWQQGNVFSCSVLHEALHSR |
| | | YTQKSLSLSPGK |
| 23 | Anti-PD-L1 LC | DIQMTQSPSSLSASVGDRVTITCRASQDVSTAVAWYQQKPGKAPKLLIYS |
| | (anti-OAcGD2 | ASFLYSGVPSRFSGSGSGTDFTLTISSLQPEDFATYYCQQYLYHPATFGQG |
| | anti-PD-L1 | TKVEIKASTKGPSVFPLAPSSKSTSGGTAALGCLVKDYFPEPVTVSWNSG |
| | bispecific) | ALTSGVHTFPAVLQSSGLYSLSSVVTVPSSSLGTQTYICNVNHKPSNTKV |
| | | DKRVEPKSC |
| 24 | Anti-DOTA | EVMLVESGGGLVKPGGSLTLSCAASGFTFTAHAMSWVRQTPEKRLEWV |
| | HC | ATISGGGTYTYFPDSFQGRFTISSDNAKNTLYLQMSSLRSEDTSMYFCTR |
| | (anti-DOTA | HGDYRYAFGYWGQGTLVTVSAASTKGPSVFPLAPSSKSTSGGTAALGCL |
| | anti-PD-L1 | VKDYFPEPVTVSWNSGALTSGVHTFPAVLQSSGLYSLSSVVTVPSSSLGT |
| | bispecific) | QTYICNVNHKPSNTKVDKRVEPKSCDKTHTCPPCPAPELLGGPDVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPEEKTISKAKG |
| | | QPREPQVYTLPPCREEMTKNQVSLWCLVKGFYPSDIAVEWESNGQPENN |
| | | YKTTPPVLDSDGSFFLYSKLTVDKSRWQQGNVFSCSVLHEALHSHYTQK |
| | | SLSLSPGK |
| 25 | Anti-DOTA LC | QAVTQESALTTSPGETVTLTCRSSSGAVTTNNYANWVQEKPDHLFTGLIG |
| | (anti-DOTA | GTNNRAPGVPARFSGSLIGDKAALTITGAQTEDEAIYFCALWFSDHWVFG |
| | anti-PD-L1 | GGTELTVLGRTVAAPSVFIFPPSDEQLKSGTASVVCLLNNFYPREAKVQW |
| | bispecific) | KVDNALQSGNSQESVTEQDSKDSTYSLSSTLTLSKADYEKHKVYACEVT |
| | | HQGLSSPVTKSFNRGEC |
| 26 | Anti-PD-L1 HC | EVQLVESGGGLVQPGGSLRLSCAASGFTFSDSWIHWVRQAPGKGLEWV |
| | (anti-DOTA | AWISPYGGSTYYADSVKGRFTISADTSKNTAYLQMNSLRAEDTAVYYCA |
| | anti-PD-L1 | RRHWPGGFDYWGQGTLVTVSSRTVAAPSVFIFPPSDEQLKSGTASVVCL |
| | bispecific) | LNNFYPREAKVQWKVDNALQSGNSQESVTEQDSKDSTYSLSSTLTLSKA |
| | | DYEKHKVYACEVTHQGLSSPVTKSFNRGECDKTHTCPPCPAPELLGGPD |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPEEKTIS |
| | | KAKGQPREPQVCTLPPSREEMTKNQVSLSCAVKGFYPSDIAVEWESNGQ |
| | | PENNYKTTPPVLDSDGSFFLVSKLTVDKSRWQQGNVFSCSVLHEALHSR |
| | | YTQKSLSLSPGK |
| 27 | Anti-PD-L1 LC | DIQMTQSPSSLSASVGDRVTITCRASQDVSTAVAWYQQKPGKAPKLLIYS |
| | (anti-DOTA | ASFLYSGVPSRFSGSGSGTDFTLTISSLQPEDFATYYCQQYLYHPATFGQG |
| | anti-PD-L1 | TKVEIKASTKGPSVFPLAPSSKSTSGGTAALGCLVKDYFPEPVTVSWNSG |
| | bispecific) | ALTSGVHTFPAVLQSSGLYSLSSVVTVPSSSLGTQTYICNVNHKPSNTKV |
| | | DKRVEPKSC |
| 28 | Anti-OAcGD2 | EVQLVESGGGLVQPGGSLRLSCATSEFTFSDYYMTWVRQAPGKGLEWL |
| | HC | GFIRNKANSYTTEYNPSVKGRFTISRDNSKNSLYLQMNSLKTEDTAVYYC |
| | (anti-OAcGD2 | ARVSNWAFDYWGQGTLVTVSSASTKGPSVFPLAPSSKSTSGGTAALGCL |
| | anti-DOTA | VKDYFPEPVTVSWNSGALTSGVHTFPAVLQSSGLYSLSSVVTVPSSSLGT |
| | bispecific) | QTYICNVNHKPSNTKVDKRVEPKSCDKTHTCPPCPAPELLGGPDVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPEEKTISKAKG |
| | | QPREPQVYTLPPCREEMTKNQVSLWCLVKGFYPSDIAVEWESNGQPENN |
| | | YKTTPPVLDSDGSFFLYSKLTVDKSRWQQGNVFSCSVLHEALHSHYTQK |
| | | SLSLSPGK |
| 29 | Anti-OAcGD2 | DVVMTQSPLSLPVTLGQPASISCRSSQSLLKNNGNTFLHWYQQRPGQSPR |
| | LC | LLIYKVSNRLSGVPDRFSGSGSGTDFTLKISRVEAEDVGVYFCSQSTHIPY |
| | (anti-OAcGD2 | TFGGGTKVEIKRTVAAPSVFIFPPSDEQLKSGTASVVCLLNNFYPREAKV |
| | anti-DOTA | QWKVDNALQSGNSQESVTEQDSKDSTYSLSSTLTLSKADYEKHKVYACE |
| | bispecific) | VTHQGLSSPVTKSFNRGEC |
| 30 | Anti-DOTA | EVMLVESGGGLVKPGGSLTLSCAASGFTFTAHAMSWVRQTPEKRLEWV |
| | HC | ATISGGGTYTYFPDSFQGRFTISSDNAKNTLYLQMSSLRSEDTSMYFCTR |
| | (anti-OAcGD2 | HGDYRYAFGYWGQGTLVTVSARTVAAPSVFIFPPSDEQLKSGTASVVCL |
| | anti-DOTA | LNNFYPREAKVQWKVDNALQSGNSQESVTEQDSKDSTYSLSSTLTLSKA |
| | bispecific) | DYEKHKVYACEVTHQGLSSPVTKSFNRGECDKTHTCPPCPAPELLGGPD |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPEEKTIS |
| | | KAKGQPREPQVCTLPPSREEMTKNQVSLSCAVKGFYPSDIAVEWESNGQ |
| | | PENNYKTTPPVLDSDGSFFLVSKLTVDKSRWQQGNVFSCSVLHEALHSR |
| | | YTQKSLSLSPGK |
| 31 | Anti-DOTA LC | QAVTQESALTTSPGETVTLTCRSSSGAVTTNNYANWVQEKPDHLFTGLIG |
| | (anti-OAcGD2 | GTNNRAPGVPARFSGSLIGDKAALTITGAQTEDEAIYFCALWFSDHWVFG |
| | anti-DOTA | GGTELTVLGASTKGPSVFPLAPSSKSTSGGTAALGCLVKDYFPEPVTVSW |
| | bispecific) | NSGALTSGVHTFPAVLQSSGLYSLSSVVTVPSSSLGTQTYICNVNHKPSNT |
| | | KVDKRVEPKSC |
| 32 | Anti-OAcGD2 | EVQLVESGGGLVQPGGSLRLSCATSEFTFSDYYMTWVRQAPGKGLEWL |
| | HC | GFIRNKANSYTTEYNPSVKGRFTISRDNSKNSLYLQMNSLKTEDTAVYYC |
| | (monospecific) | ARVSNWAFDYWGQGTLVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPDVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPEEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVLHEALHSHYTQK |
| | | SLSLSPGK |
| 33 | Anti-OAcGD2 | DVVMTQSPLSLPVTLGQPASISCRSSQSLLKNNGNTFLHWYQQRPGQSPR |
| | LC | LLIYKVSNRLSGVPDRFSGSGSGTDFTLKISRVEAEDVGVYFCSQSTHIPY |
| | (monospecific) | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACE |
| | | VTHQGLSSPVTKSFNRGEC |
| 34 | Anti-DOTA | EVMLVESGGGLVKPGGSLTLSCAASGFTFTAHAMSWVRQTPEKRLEWV |
| | HC | ATISGGGTYTYFPDSFQGRFTISSDNAKNTLYLQMSSLRSEDTSMYFCTR |
| | (monospecific) | HGDYRYAFGYWGQGTLVTVSAASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPDVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPEEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVLHEALHSHYTQK |
| | | SLSLSPGK |
| 35 | Anti-DOTA LC | QAVTQESALTTSPGETVTLTCRSSSGAVTTNNYANWVQEKPDHLFTGLIG |
| | (monospecific) | GTNNRAPGVPARFSGSLIGDKAALTITGAQTEDEAIYFCALWFSDHWVFG |
| | | GGTELTVLGRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| 36 | Anti-PD-L1 HC | EVQLVESGGGLVQPGGSLRLSCAASGFTFSDSWIHWVRQAPGKGLEWV |
| | (monospecific) | AWISPYGGSTYYADSVKGRFTISADTSKNTAYLQMNSLRAEDTAVYYCA |
| | | RRHWPGGFDYWGQGTLVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPDVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPEEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVLHEALHSHYTQK |
| | | SLSLSPGK |
| 37 | Anti-PD-L1 LC | DIQMTQSPSSLSASVGDRVTITCRASQDVSTAVAWYQQKPGKAPKLLIYS |
| | (monospecific) | ASFLYSGVPSRFSGSGSGTDFTLTISSLQPEDFATYYCQQYLYHPATFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 38 | Anti-PD-L1 | GFTFSDSW |
| | H-CDR1 | |
| 39 | Anti-PD-L1 | ISPYGGST |
| | H-CDR2 | |
| 40 | Anti-PD-L1 | ARRHWPGGFDY |
| | H-CDR3 | |
| 41 | Anti-PD-L1 | QDVSTA |
| | L-CDR1 | |
| null | Anti-PD-L1 | SAS |
| | L-CDR2 | |
| 43 | Anti-PD-L1 | QQYLYHPAT |
| | L-CDR3 | |
| 44 | Anti-PD-L1 | EVQLVESGGGLVQPGGSLRLSCAASGFTFSDSWIHWVRQAPGKGLEWV |
| | VH | AWISPYGGSTYYADSVKGRFTISADTSKNTAYLQMNSLRAEDTAVYYCA |
| | | RRHWPGGFDYWGQGTLVTVSS |
| 45 | Anti-PD-L1 | DIQMTQSPSSLSASVGDRVTITCRASQDVSTAVAWYQQKPGKAPKLLIYS |
| | VL | ASFLYSGVPSRFSGSGSGTDFTLTISSLQPEDFATYYCQQYLYHPATFGQG |
| | | TKVEIK |

### Quality Control of the Antibodies

Purity was assessed via SDS-PAGE, analytical size-exclusion chromatography, and endotoxin tests.

### Bispecific Binding Using BioLayer Interferometry (BLI)

Bispecific binding was evaluated by BLI technology (Octet BLItZ) on immobilized purified PD-L1 protein and OAcGD2. BLI analysis was performed on Octet R8 or R2 instrument using standard streptavidin (SA) biosensors or standard NiNTA biosensors. PBS was used as loading buffer. OAcGD2 sugar (OAcGD2s) loading was performed by loading biotinylated OAcGD2s (used at 0.25 μg/mL in PBS) to SA-biosensors for 60 seconds. PD-L1 loading was performed by loading His-tagged PD-L1 (used at 200 nM in PBS) to NiNTA-biosensors for 60 seconds. After loading, biosensors were washed in standard assay buffer (PBS-BSA 0.1%-Tween20 0.02%) until a stable baseline was obtained. Antibody binding was performed by moving OAcGD2s-loaded biosensors or PD-L1-loaded biosensors to wells containing antibody samples at the concentration of 100 nM in standard assay buffer. Then, the antibody-loaded biosensors were moved to standard assay buffer to examine antibody dissociation. Data were exported from the Octet Analysis Studio 12.2 software into excel files to confirm the binding of the antibodies to OAcGD2s or PD-L1 (No kinetics were derived from these experiments).

### In Vitro Studies

### Cell Culture

Mouse neuroblastoma NXS2 cell line was kindly provided by Dr. H. N. Lode (Universitätsklinikum Greifswald, Greifswald, Germany). NXS2 cells were grown in DMEM supplemented with 10% heat-inactivated fetal calf serum, 2 mM L-Glutamin, 100 units/mL penicillin, and 100 μg/mL streptomycin, at 37° C. in a humidified incubator at 5% CO2 atmosphere. Human neuroblastoma LAN-1 cell line (DSMZ GmbH, Germany) were grown in RPMI 1640 supplemented with 10% heat-inactivated fetal calf serum, 2 mM L-Glutamin, 100 units/mL penicillin, and 100 μg/mL streptomycin, at 37° C. in a humidified incubator at 5% CO2 atmosphere.

### Calcein Release ADCC Assay

Human neuroblastoma LAN-1 cells were labelled with Calcein-AM for 30 min, then washed and plated at a density of 5000 cells/well onto U bottom 96-well plates. Varying concentrations of the OAcGD2/PD-L1 bispecific antibody and control monospecific antibodies were added and peripheral blood mononuclear cells (PBMCs) were added to the LAN-1 cells wells at 1×10⁵ cells/well and incubated for 2 h (an effector:target ratio of 20:1) at 37° C. The supernatants were analyzed using fluorometry (on a Fluostar Omega Reader) to measure calcein release (cell death). For maximal release, the cells were lysed with 2% Triton X-100. The fluorescence value of the culture medium background was subtracted from that of the experimental release (A), the target cell spontaneous release (B) and the target cell maximal release (C). The cytotoxicity and ADCC percentages for each plate (in triplicate) were calculated using the following formulas:

$$Cytotoxicity ( \% ) = ( A - B ) / ( C - B ) \times 100$$

$$ADCC ( \% ) = Cytotoxicity ( \% , with antibody ) - Cytotoxicity ( \% , without antibody )$$

### ADCC Reporter Bioassay

This experiment was performed using Promega ADDC Reporter Bioassay kit (Ref.

G7010). Human neuroblastoma LAN-1 cells were plated onto white 96-well plates at a density of 12 500 cells/well and allow to grow in fresh culture medium for 20 to 24 hours. Twenty-four hours later, medium was replaced by ADCC Bioassay buffer (RPMI 1640 containing low IgG serum). Series of concentrations for the OAcGD2/PD-L1 bispecific antibody and control monospecific antibodies ranging from 10 to 0 μg/ml were added. ADCC Bioassay effector cells (Jurkat cells stably expressing the FcγRIIIa receptor, V₁₅₈ and NFAT pathway quantified through firefly luciferase production) were then added to the LAN-1 cells wells at 7.5×10⁵ cells/well and incubated for 6 h (an effector:target ratio of 6:1) at 37° C. Six hours after, plates were allowed to equilibrate to ambient temperature (22-25° C.) during 15 minutes, and Bio-Glo™ Luciferase Assay Reagent was added to all wells and incubated at ambient temperature for 5-30 minutes. Luminescence was measured using a Fluostar Omega plate Reader. Luminescence value of the culture medium background was subtracted from that of the experimental Relative Light Units (RLU) values. Graph data were plotted as RLU versus Log 10 [antibody concentration] and fit curves to calculate EC50 of antibodies were calculated using GraphPad Prism software.

### In Vivo Studies

Anti-neuroblastoma efficacy of anti-OAcGD2, anti-PD-L1 and anti-OAcGD2/Anti-PD-L1 bispecific antibody treatments was determined in the murine NXS2 neuroblastoma experimental liver metastasis model in A/J mice.

### Anti-Tumoral Efficacy Study of Repeated Doses

Animals: 10-weeks old female A/J mice (Jackson Laboratories, US). Ten mice were assigned per group of treatment. Mice were housed at the UTE-IRS2 animal facility (Nantes, France). All in vivo experiments were carried out in strict accordance with the recommendations in the Guide for the Care and Use of Laboratory Animals of the French Department of Agriculture. The protocol was approved by the Committee on the Ethics of Animal Experiments of the Région Pays de la Loire (permit number: 35513v4).

Engraftment: NXS2 Cells (2.5×10⁵ cells in 100 μL of non-supplemented DMEM medium) were transplanted i.v. into syngeneic 10-weeks old female A/J mice, to induce liver metastasis.

Treatment schedule: Mice received either anti-OAcGD2 antibody, or PD-L1 blocking antibody, or OAcGD2/PD-L1 bispecific antibody, or anti-DOTA mAb as isotypic control alone as single agent therapy or the combination regimen for anti-OAcGD2 and anti-PD-L1 antibodies twice a week for 3 consecutive weeks beginning 3 days after tumor cell engraftment. We assigned 10 mice to treatment with either the single agent therapy (50 μg per injection i.v.; 300 μg total dose injection), or the combination regimen for anti-OAcGD2 and anti-PD-L1 antibodies (25 μg for each mAb mixed per injection; 300 μg mAb total dose injection). Mice were euthanized on day 28 post-tumor challenge, and anti-tumor efficacy was evaluated by determining the liver weight of fresh specimen and the number of liver metastasis.

### Dose-Response Study

Animals: 10-weeks old female A/J mice (Jackson Laboratories, US). Ten mice were assigned per group of treatment. Mice were housed at the UTE-IRS2 animal facility (Nantes, France). All in vivo experiments were carried out in strict accordance with the recommendations in the Guide for the Care and Use of Laboratory Animals of the French Department of Agriculture. The protocol was approved by the Committee on the Ethics of Animal Experiments of the Région Pays de la Loire (permit number: 35513v4).

Engraftment: NXS2 Cells (2.5×10⁵ cells in 100 μL of non-supplemented DMEM medium) were transplanted i.v. into syngeneic 10-weeks old female A/J mice, to induce liver metastasis.

Treatment schedule: The single agent therapy was evaluated in a dose response experiment with following protocol. Mice received either anti-OAcGD2 antibody, or OAcGD2/PD-L1 bispecific antibody, or anti-DOTA mAb as isotypic control at a dose of 50 μg per injection i.v. (300 μg total dose injection), 16.67 μg per injection i.v. (100 μg total dose injection), 5 μg per injection i.v. (30 μg total dose injection) or 1.67 μg per injection i.v. (10 μg total dose injection). We assigned 10 mice per group of treatment. Mice were euthanized on day 28 post-tumor challenge, and anti-tumor efficacy was evaluated by determining the liver weight of fresh specimen and the number of liver metastasis.

### REFERENCES

Throughout this application, various references describe the state of the art to which this invention pertains. The disclosures of these references are hereby incorporated by reference into the present disclosure.
1. Nakagawara A, Ohira M. Comprehensive genomics linking between neural development and cancer: neuroblastoma as a model. Cancer Lett 2004; 204: 213-24.
2. Katzenstein H M, Bowman L C, Brodeur G M, Thorner P S, Joshi V V, Smith E I, Look A T, Rowe S T, Nash M B, Holbrook T, Alvarado C, Rao P V, et al. Prognostic significance of age, MYCN oncogene amplification, tumor cell ploidy, and histology in 110 infants with stage D(S) neuroblastoma: the pediatric oncology group experience—a pediatric oncology group study. J Clin Oncol 1998; 16: 2007-17.
3. Cohn S L, Pearson A D, London W B, Monclair T, Ambros P F, Brodeur G M, Faldum A, Hero B, Iehara T, Machin D, Mosseri V, Simon T, et al. The International Neuroblastoma Risk Group (INRG) classification system: an INRG Task Force report. J Clin Oncol 2009; 27: 289-97.
4. Yu A L, Gilman A L, Ozkaynak M F, London W B, Kreissman S G, Chen H X, Smith M, Anderson B, Villablanca J G, Matthay K K, Shimada H, Grupp S A, et al. Anti-GD2 antibody with G M-CSF, interleukin-2, and isotretinoin for neuroblastoma. N Engl J Med 2010; 363: 1324-34.
5. Ladenstein R, Potschger U, Valteau-Couanet D, Luksch R, Castel V, Yaniv I, Laureys G, Brock P, Michon J M, Owens C, Trahair T, Chan GCF, et al. Interleukin 2 with anti-GD2 antibody ch14.18/CHO (dinutuximab beta) in patients with high-risk neuroblastoma (HR-NBL1/SIOPEN): a multicentre, randomised, phase 3 trial. Lancet Oncol 2018; 19: 1617-29.
6. Zeng Y, Fest S, Kunert R, Katinger H, Pistoia V, Michon J, Lewis G, Ladenstein R, Lode H N. Anti-neuroblastoma effect of ch14.18 antibody produced in CHO cells is mediated by NK-cells in mice. Mol Immunol 2005; 42: 1311-9.
7. Siebert N, Jensen C, Troschke-Meurer S, Zumpe M, Juttner M, Ehlert K, Kietz S, Muller I, Lode H N. Neuroblastoma patients with high-affinity FCGR2A, -3A and stimulatory KIR 2DS2 treated by long-term infusion of anti-GD2 antibody ch14.18/CHO show higher ADCC levels and improved event-free survival. Oncoimmunology 2016; 5: e1235108.
8. Henney C S, Kuribayashi K, Kern D E, Gillis S. Interleukin-2 augments natural killer cell activity. Nature 1981; 291: 335-8.
9. Lanier L L. N K cell recognition. Annu Rev Immunol 2005; 23: 225-74.
10. Sanchez-Correa B, Lopez-Sejas N, Duran E, Labella F, Alonso C, Solana R, Tarazona R. Modulation of N K cells with checkpoint inhibitors in the context of cancer immunotherapy. Cancer Immunol Immunother 2019; 68: 861-70.
11. van Dam L S, de Zwart V M, Meyer-Wentrup F A. The role of programmed cell death-1 (PD-1) and its ligands in pediatric cancer. Pediatr Blood Cancer 2015; 62: 190-7.
12. Benson D M, Jr., Bakan C E, Mishra A, Hofmeister C C, Efebera Y, Becknell B, Baiocchi R A, Zhang J, Yu J, Smith M K, Greenfield C N, Porcu P, et al. The PD-1/PD-L1 axis modulates the natural killer cell versus multiple myeloma effect: a therapeutic target for CT-011, a novel monoclonal anti-PD-1 antibody. Blood 2010; 116: 2286-94.
13. Siebert N, Zumpe M, Juttner M, Troschke-Meurer S, Lode H N. PD-1 blockade augments anti-neuroblastoma immune response induced by anti-GD2 antibody ch14.18/CHO. Oncoimmunology 2017; 6: e1343775.
14. Helson L, Helson C. Human neuroblastoma cells and 13-cis-retinoic acid. J Neurooncol 1985; 3: 39-41.
15. Abemayor E, Chang B, Sidell N. Effects of retinoic acid on the in vivo growth of human neuroblastoma cells. Cancer Lett 1990; 55: 1-5.
16. Jin H J, Nam H Y, Bae Y K, Kim S Y, Im I R, Oh W, Yang Y S, Choi S J, Kim S W. GD2 expression is closely associated with neuronal differentiation of human umbilical cord blood-derived mesenchymal stem cells. Cell Mol Life Sci 2010; 67: 1845-58.
17. Erkelens M N, Mebius R E. Retinoic Acid and Immune Homeostasis: A Balancing Act. Trends Immunol 2017; 38: 168-80.
18. Nguyen R, Houston J, Chan W K, Finkelstein D, Dyer M A. The role of interleukin-2, all-trans retinoic acid, and natural killer cells: surveillance mechanisms in anti-GD2 antibody therapy in neuroblastoma. Cancer Immunol Immunother 2018; 67: 615-26.
19. Li A, He M, Wang H, Qiao B, Chen P, Gu H, Zhang M, He S. All-trans retinoic acid negatively regulates cytotoxic activities of nature killer cell line 92. Biochem Biophys Res Commun 2007; 352: 42-7.
20. Alvarez-Rueda N et al. A monoclonal antibody to O-acetyl-GD2 ganglioside and not to GD2 shows potent anti-tumor activity without peripheral nervous system cross-reactivity. PLOS One 2011; 6: e25220.
21. Cochonneau D et al. Cell cycle arrest and apoptosis induced by O-acetyl-GD2-specific monoclonal antibody 8B6 inhibits tumor growth in vitro and in vivo. Cancer Lett 2013; 333: 194-204.
22. Terme M et al. Chimeric antibody c.8B6 to O-acetyl-GD2 mediates the same efficient anti-neuroblastoma effects as therapeutic ch14.18 antibody to GD2 without antibody induced allodynia. PLOS One 2014; 9: e87210.
23. Nair A B, Jacob S. A simple practice guide for dose conversion between animals and human. J Basic Clin Pharm 2016; 7: 27-31.
24. Ullman-Cullere M H, Foltz C J. Body condition scoring: a rapid and accurate method for assessing health status in mice. Lab Anim Sci 1999; 49: 319-23.
25. Iorgulescu J B, Braun D, Oliveira G, Keskin D B, Wu C J. Acquired mechanisms of immune escape in cancer following immunotherapy. Genome Med 2018; 10: 87.
26. Hsu J, et al. Contribution of N K cells to immunotherapy mediated by PD-1/PD-L1 blockade. J Clin Invest 2018; 128: 4654-68.
27. Fleurence Julien et al. 2016. Targeting and killing glioblastoma with monoclonal antibody to O-acetyl GD2 ganglioside. Oncotarget.
28. Lode H N et al. Targeted interleukin-2 therapy for spontaneous neuroblastoma metastases to bone marrow. J Natl Cancer Inst 1997; 89: 1586-94.
29. Federico Sara M. et al. A Pilot Trial of Humanized Anti-GD2 Monoclonal Antibody (hu14.18K322A) with Chemotherapy and Natural Killer Cells in Children with Recurrent/Refractory Neuroblastoma. Clin Cancer Res; 23(21) Nov. 1, 2017.
30. Modak Shakeel et al. Adoptive immunotherapy with haploidentical natural killer cells and Anti-GD2monoclonal antibody m3F8 for resistant neuroblastoma: Results of a phase I study. Oncoimmunology 2018, VOL. 7, NO. 8, e1461305.

1-15. (canceled)

16. A method for treating a cancer comprising administering to a subject in need thereof a pharmaceutical composition comprising retinoic acid, an anti-O-acetylated disialoganglioside (OAcGD2) compound and an anti-PD1 or anti-PD-L1 compound.

17. The method according to claim 16, wherein said pharmaceutical composition further comprises at least one anti-cancer agent.

18. A method for treating a cancer comprising administering to a subject in need thereof a therapeutically effective amount of retinoic acid, an anti-O-acetylated disialoganglioside (OAcGD2) compound and an anti-PD1 or anti-PD-L1 compound.

19. The method according to claim 18, wherein the retinoic acid is 13-cis-retinoic acid.

20. The method according to claim 18, wherein the cancer is a neuroblastoma, a glioblastoma, a small-cell lung carcinoma or a breast cancer.

21. The method according to claim 18, wherein the anti-OAcGD2 compound is an antibody comprising the following complementary-determining regions (CDRs):
CDR1:
(SEQ ID NO: 1)
EFTFTDYY;
CDR2:
(SEQ ID NO: 2)
IRNRANGYTT;
CDR3:
(SEQ ID NO: 3)
ARVSNWAFDY;
CDR4:
(SEQ ID NO: 4)
QSLLKNNGNTFL;
CDR5:
(SEQ ID NO: 5)
KVS;
and
CDR6:
(SEQ ID NO: 6)
SQSTHIPYT.

22. The method according to claim 18, wherein the anti-OAcGD2 compound is a humanized antibody.

23. The method according to claim 18, wherein the anti-OAcGD2 compound is an antibody comprising:
a) a light chain variable region (VL) having the amino acid sequence SEQ ID NO: 7, or a sequence having a percentage of identity of at least 85% with SEQ ID NO: 7; and
b) a heavy chain variable region (VH) having the amino acid sequence SEQ ID NO: 8, or a sequence having a percentage of identity of at least 85% with SEQ ID NO: 8.

24. The method according to claim 18, wherein the anti-OAcGD2 compound is an antibody having a sequence comprising:
a heavy chain variable region (VH) sequence selected from the group consisting of SEQ ID NO: 9 (“VH49A”), SEQ ID NO: 10 (“VH72A”), SEQ ID NO: 11 (“VH49BHS”), SEQ ID NO: 12 (“VH72BHNPS”), SEQ ID NO: 16 (“VH49BHSs”), SEQ ID NO: 17 (“VH72BHNPSs”), or a variant thereof; and/or
a light chain variable region (VL) sequence selected from the group consisting of SEQ ID NO: 13 (“VL30A”), SEQ ID NO: 14 (“VL28A”), SEQ ID NO: 15 (“VL28Bs01/A2”), SEQ ID NO: 18 (“VL30As”), SEQ ID NO: 19 (“VL28B01/A2”), or a variant thereof.

25. The method according to claim 18, wherein the anti-O-acetylated disialoganglioside (OAcGD2) compound and the anti-PD1 or anti-PD-L1 compound are a multi-specific antibody having at least two antigen binding sites directed to OAcGD2 and to PD1 or PD-L1.

26. The method according to claim 25, wherein the multi-specific antibody is a bi-specific antibody directed to OAcGD2 and to PD1 or PD-L1, or a derivative thereof.

27. The method according to claim 25, wherein the multi-specific antibody is a trivalent or tetravalent antibody comprising at least two antigen binding sites directed to OAcGD2 and at least one antigen binding site directed to PD1 or PD-L1.

28. A method for treating a cancer comprising administering to a subject in need thereof a therapeutically effective amount of allogenic NK cells treated with retinoic acid, an anti-O-acetylated disialoganglioside (OAcGD2) compound and an anti-PD1 or anti-PD-L1 compound.

29. The method according to claim 17, wherein the at least one anti-cancer agent is interleukin-2 (IL-2) and/or an anti-disialoganglioside (anti-GD2) antibody.

30. The method according to claim 18, wherein the cancer is a neuroblastoma.

31. The method according to claim 18, wherein retinoic acid, the anti-O-acetylated disialoganglioside (OAcGD2) compound and the anti-PD1 or anti-PD-L1 compound are administered simultaneously, separately, or sequentially.
